(19) United States
(12) Patent Application Publication
(10) Pub. No.: US 2010/0330122 A1
(43) Pub. Date: Dec. 30, 2010

(54) VARICELLA ZOSTER VIRUS VIRUS-LIKE PARTICLES (VLPs) AND ANTIGENS

(76) Inventors: Gale Smith, Rockville, MD (US); Peter Pushko, Frederick, MD (US)

Correspondence Address: COOLEY LLP;ATTN: Patent Group, Suite 1100, 777 - 6th Street, NW, WASHINGTON, DC, 20001, US

(21) Appl. No.: 12/669,700

(22) PCT Filed: Jul. 21, 2008
(86) PCT No.: PCT/US08/70635

§ 371 Date: Sep. 10, 2010

Related U.S. Application Data
(60) Provisional application No. 60/950,707, filed on Jul. 19, 2007.

(51) Int. Cl. A61K 39/25; C07K 14/04; A61P 37/04

(52) U.S. Cl. 424/229.1; 530/350

(57) ABSTRACT

The present invention discloses novel Varicella Zoster Virus (VZV) virus-like particles (VLPs) comprising glycoprotein E of VZV. The invention also discloses vaccine formulations of the VZV-VLPs and methods of inducing an immune response in subjects.

This application claims priority to U.S. application 60/950,707, filed Jul. 19, 2007, which is herein incorporated by reference in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing of the Sequence Listing (filename: NOVV 019 01 WO SeqList_ST25.txt, date recorded: Jul. 21, 2008, file size 106 kilobytes).

### BACKGROUND OF THE INVENTION

The Varicella Zoster virus (VZV), also known as human herpesvirus 3 (HHV-3), is a member of the alphaherpesvirus subfamily of the Herpesviridae family of viruses. VZV is an enveloped virus with a linear double-stranded DNA genome of approximately 125, 000 nucleotides. Its genome is encased by an icosahedral nucleocapsid. The tegument, located in the space between the nucelocapsid and the viral envelope, is a structure comprised of virally-encoded proteins and enzymes. The viral envelope is acquired from host cell membranes and contains viral-encoded glycoproteins.

The VZV genome, the smallest among the human herpesviruses, encodes at least 70 open reading frames, eight of which encode putative glycoproteins (gE, gI, gB, gH, gK, gL, gC, and gM) that function in different steps of the viral replication cycle. Glycoprotein E (gE, also designated ORF 68) is essential for viral replication (Mallory et al. (1997) J. Virol. 71: 8279-8288; Mo et al. (2002) Virology 304: 176-186), and is the most abundant glycoprotein found in infected cells as well as the mature virion (Grose, 2002, The predominant varicella-zoster virus gE and gI glycoprotein complex, In Structure-function relationships of human pathogenic viruses, Holzenburg and Bogner (eds.), Kluwer Academic/Plenum Publishers, New York, N.Y.). Glycoprotein I (gI, also designated ORF 67) forms a complex with gE in infected cells, which facilitates the endocytosis of both glycoproteins and directs them to the trans-Golgi where the final viral envelope is acquired (Olson and Grose (1998) J. Virol. 72:1542-1551). Glycoprotein B (gB, also designated ORF 31), thought to play a role in virus entry, binds to neutralizing antibodies and is the second most prevalent glycoprotein (reviewed in Arvin (1996) Clin. Microbiol. Rev. 9: 361-381). Glycoprotein H (gH) is thought to have a fusion function facilitating cell to cell spread of the virus. Antibodies to gE, gB, and gH are prevalent after natural infection and following vaccination, and have been shown to neutralize viral infectivity in vitro (Keller et al. (1984) J. Virol. 52: 293-297; Arvin et al. (1986) J. Immunol. 137: 1346-1351; Vafai et al. (1988) J. Virol. 62: 2544-2551; Forghani et al. (1990) J. Clin. Microbiol. 28: 2500-2506).

Primary infection with VZV causes chickenpox (varicella) characterized by an extremely contagious skin rash occurring predominantly on the face and trunk. After initial infection, the viral DNA can integrate into the genome of host neuronal cells and remain dormant for several years. The virus can reactivate and cause the disease shingles (herpes zoster) in adults. Shingles produces a skin rash that is distinct from that produced during the primary infection. The rash is associated with severe pain and can result in more serious conditions, such as post-herpetic neuralgia.

A Varicella vaccine (Varivax) is available to the general public and has been added to the recommended vaccination schedule for children in several countries including the United States. A more concentrated formulation of the Varicella vaccine (Zostavax) has been approved by the Food and Drug Administration for prevention of shingles in older members of the population. Although the Varicella vaccine has proven to be safe, there is some evidence that the immunity to VZV infection conferred by the vaccine wanes over time (Chaves et al. (2007) N. Engl. J. Med. 356: 1121-1129). Therefore, vaccinated individuals would remain susceptible to shingles, the more serious condition caused by VZV. In addition, the vaccine is made from live attenuated virus, which creates the possibility of an individual developing either chickenpox or shingles from the vaccination. In fact, there have been several cases of shingles reported that appeared to be caused by the strain used in the vaccine (Matsubara et al. (1995). Acta Paediatr Jpn 37: 648-50; Hammerschlag et al. (1989). J Infect Dis. 160: 535-7). The live attenuated virus present in the vaccine also limits the use of the vaccine in immunocompromised individuals.

Virus-like particles (VLPs) are structurally similar to mature virions, but lack the viral genome making it impossible for viral replication to occur. VLPs contain antigenic proteins, such as capsid and viral envelope proteins, like intact virus and can be constructed to express foreign structural proteins on their surface as well. Therefore, VLPs can be administered safely as an immunogenic composition (e.g. vaccine). Furthermore, since VLPs resemble the native virus and are multivalent particulate structures, VLPs may be more effective in inducing neutralizing antibodies than soluble antigens.

VLPs expressing glycoproteins or tegument proteins have previously been generated from different herpesvirus family members. Light particles (L-particles) comprised of enveloped tegument proteins, have been obtained from cells infected with either herpes simplex virus type 1 (HSV-1), equine herpesvirus type 1 (EHV-1), or pseudorabies virus (McLauchlan and Rixon (1992) J. Gen. Virol. 73: 269-276; U.S. Pat. No. 5,384,122). A different type of VLP, termed pre-viral DNA replication enveloped particles (PREPs), could be generated from cells infected with HSV-I in the presence of viral DNA replication inhibitors. The PREPs resembled L-particles structurally, but contained a distinct protein composition (Dargan et al. (1995) J. Virol. 69: 4924-4932; U.S. Pat. No. 5,994,116). Hybrid VLPs expressing fragments of the gE protein from VZV have been produced by a technique using protein p1 encoded by the yeast Ty retrotransposon (Garcia-Valcarcel et al. (1997) Vaccine 15: 709-719; Welsh et al. (1999) J. Med. Virol. 59: 78-83; U.S. Pat. No. 6,060,064). The present invention describes novel antigens and VLPs derived from VZV that do not require expression of a yeast Ty protein nor infection with the virus itself. These novel VLPs are useful as antigenic formulations or vaccine preparations.

### SUMMARY OF THE INVENTION

The present invention comprises a purified virus like particle (VLP) from Varicella Zoster Virus (VZV) comprising VZV gE protein, but does not include VZV nucleic acid or a yeast Ty protein. In one embodiment, said VZV-VLP further comprises at least one additional protein from an infectious agent. In another embodiment, said additional protein from an infectious agent is from a virus. In another embodiment, said additional protein from an infectious agent is from a fungus. In another embodiment, said additional protein from an infectious agent is from a parasite. In another embodiment, said additional protein from an infectious agent is from a bacterium. In another embodiment, said additional protein from an infectious agent is expressed on the surface of the VZV-VLP. In another embodiment, said VZV-VLP consists essentially of VZV gE. In another embodiment, said VZV-VLP is derived from a recombinant expression system comprising a cloned gE VZV.

The present invention also includes a purified VZV-VLP comprising VZV gE protein and an additional VZV protein, but does not contain VZV nucleic acid or a yeast Ty protein. In one embodiment, said additional VZV protein is gI (ORF 67). In another embodiment, said additional VZV protein is gM (ORF 50). In another embodiment, said additional VZV protein is gH. In another embodiment, said additional VZV protein is gB. In another embodiment, said additional VZV protein is a tegument protein. In another embodiment, said VZV-VLP further comprises an additional protein from an infectious agent.

The present invention also provides a method of producing a VLP, comprising transfecting a vector encoding VZV gE protein into a suitable host cell and expressing said VZV gE protein under conditions that allow VLPs to be formed, isolated and/or purified, wherein said host cell does not comprise a yeast Ty protein and said VLP does not comprise VZV nucleic acid. In one embodiment, said VZV-VLP further comprises at least one additional protein from an infectious agent. In another embodiment, said additional protein from an infectious agent is from a virus. In another embodiment, said additional protein from an infectious agent is from a fungus. In another embodiment, said additional protein from an infectious agent is from a parasite. In another embodiment, said additional protein from an infectious agent is from a bacterium. In another embodiment, said additional protein from an infectious agent is expressed on the surface of the VZV-VLP. In another embodiment, said VZV-VLP consists essentially of VZV gE. In another embodiment, said host cell is a mammalian cell. In another embodiment, said host cell is an avian cell. In another embodiment, said host cell is an amphibian cell. In another embodiment, said host cell is a yeast cell. In a preferred embodiment, said host cell is an insect cell. In another preferred embodiment, said insect cells is a Sf9 cell.

The present invention also comprises a method of producing a VLP, comprising transfecting a vector encoding VZV gE protein into a suitable host cell and expressing said VZV gE protein under conditions that allow VLPs to be formed, isolated and/or purified, wherein said host cell does not comprise a yeast Ty protein and said VLP does not comprise VZV nucleic acid, and wherein said VLP further comprises an additional VZV protein. In one embodiment, said additional VZV protein is gI (ORF 67). In another embodiment, said additional VZV protein is gM (ORF 50). In another embodiment, said additional VZV protein is gH. In another embodiment, said additional VZV protein is gB. In another embodiment, said additional VZV protein is a tegument protein. In another embodiment, said VLP further comprises an additional protein from an infectious agent.

The present invention also comprises an antigenic formulation comprising a VZV-VLP, wherein said VZV-VLP comprises VZV gE and wherein said VLP does not comprise a yeast Ty protein and does not comprise VZV nucleic acid. In one embodiment, said VZV-VLP further comprises at least one additional protein from an infectious agent. In another embodiment, said additional protein from an infectious agent is from a virus. In another embodiment, said additional protein from an infectious agent is from a fungus. In another embodiment, said additional protein from an infectious agent is from a parasite. In another embodiment, said additional protein from an infectious agent is from a bacterium. In another embodiment, said additional protein from an infectious agent is expressed on the surface of the VZV-VLP. In another embodiment, said VZV-VLP consists essentially of VZV gE. In another embodiment, said antigenic formulation further comprises an adjuvant.

The present invention also comprises an antigenic formulation comprising a VZV-VLP comprising VZV gE protein and an additional VZV protein, but does not contain VZV nucleic acid or a yeast Ty protein. In one embodiment, said additional VZV protein is gI (ORF 67). In another embodiment, said additional VZV protein is gM (ORF 50). In another embodiment, said additional VZV protein is gH. In another embodiment, said additional VZV protein is gB. In another embodiment, said additional VZV protein is a tegument protein. In another embodiment, said VZV-VLP further comprises an additional protein from an infectious agent.

The present invention also includes a vaccine comprising a VZV-VLP, wherein said VZV-VLP comprises VZV gE and wherein said VLP does not comprise a yeast Ty protein and does not comprise VZV nucleic acid. In one embodiment, said VZV-VLP further comprises at least one additional protein from an infectious agent. In another embodiment, said additional protein from an infectious agent is from a virus. In another embodiment, said additional protein from an infectious agent is from a fungus. In another embodiment, said additional protein from an infectious agent is from a parasite. In another embodiment, said additional protein from an infectious agent is from a bacterium. In another embodiment, said additional protein from an infectious agent is expressed on the surface of the VZV-VLP. In another embodiment, said VZV-VLP consists essentially of VZV gE. In another embodiment, said vaccine further comprises an adjuvant.

The present invention also includes a vaccine comprising a VZV-VLP comprising VZV gE protein and an additional VZV protein, but does not contain VZV nucleic acid or a yeast Ty protein. In one embodiment, said additional VZV protein is gI (ORF 67). In another embodiment, said additional VZV protein is gM (ORF 50). In another embodiment, said additional VZV protein is gH. In another embodiment, said additional VZV protein is gB. In another embodiment, said additional VZV protein is a tegument protein. In another embodiment, said VZV-VLP further comprises an additional protein from an infectious agent.

The present invention also comprises a method of eliciting protective immunity to an infection in a human or animal comprising administering to the human or animal an antigenic formulation or vaccine comprising VZV-VLPs wherein said VZV VLPs comprise VZV gE and wherein said VLP does not comprise a yeast Ty protein and does not comprise VZV nucleic acid. In one embodiment, said VZV-VLP further comprises at least one additional protein from an infectious agent. In another embodiment, said additional protein from an infectious agent is from a virus. In another embodiment, said additional protein from an infectious agent is from a fungus. In another embodiment, said additional protein from an infectious agent is from a parasite. In another embodiment, said additional protein from an infectious agent is from a bacterium. In another embodiment, said additional protein from an infectious agent is expressed on the surface of the VZV-VLP. In another embodiment, said VZV-VLP consists essentially of VZV gE. In another embodiment, said VZV-VLP is derived from a recombinant expression system comprising a cloned gE VZV.

The present invention also comprises a method of eliciting protective immunity to an infection in a human or animal comprising administering to the human or animal an antigenic formulation or vaccine comprising VZV-VLPs wherein said VZV-VLPs comprise VZV gE protein and an additional VZV protein, but does not contain VZV nucleic acid or a yeast Ty protein. In one embodiment, said additional VZV protein is gI (ORF 67). In another embodiment, said additional VZV protein is gM (ORF 50). In another embodiment, said additional VZV protein is gH. In another embodiment, said additional VZV protein is gB. In another embodiment, said additional VZV protein is a tegument protein. In another embodiment, said VZV-VLP further comprises an additional protein from an infectious agent.

### BRIEF DESCRIPTION OF THE FIGURES

FIG. 1 depicts an SDS PAGE and two western blots of VZV expression constructs.

FIG. 2 depicts particle analysis of gE VLPs S400 Gel Filtration.

FIG. 3 depicts (A) a diagram of a pFastBac1 plasmid containing the IE62 (ICP4) tegument gene under the transcriptional control of the AcMNPV baculovirus polyhedrin promoter (PolH). (B) depicts a SDS-PAGE stained with Coomassie blue (left panel) and Western blot against anti-IE62 monoclonal antibody (right panel). Lane 1. Sample of total infected Sf9 cells expressing recombinant IE62, Lane 2. Cell lysate following homogenization and removal of cell debris by centrifugation, Lane 3. flow through of the TMAE ion exchange column, Lane 4 and 5. TMAE fractions containing IE62, Lane 6. sample loaded onto a Fractogel cation exchange sulfate (SO₃) column, Lane 7. SO3− column flow through, Lane 8. purified IE62 eluate.

FIG. 4 depicts (A) pFastBac1 transfer vector used to construct tandem recombinant baculovirus expressing VZV gE and gI in Sf9 cells. The gE and gI genes are under the transcription control of the baculovirus polyhedrin promoter (PolH). gI has its native, cleavable signal peptide and gE was replaced with the signal sequence from the baculovirus envelope glycoprotein GP64. (B) depicts SDS-PAGE gel stained with Coomassie blue (left panel) and Western blot against anti-gE (middle panel), or anti whole virus VZV (right panel). Lane 1. the load for S200 gel filtration column and Lane 2. final purified gE/gI heterodimer off the 5200 column. Anti-gE recognized the secreted gE with the major protein being the predicted mass of gE of about 60 KDa (middle panel) and polyclonal antibody reacted with a 30 KDa gI (right panel).

FIG. 5 depicts (A) pcDNA3.1 plasmid (Invitrogen) containing the C-terminal truncated VZV gE gene. (B) depicts SDS-PAGE stained with Coomassie blue (left panel) and Western blot against anti-gE monoclonal antibody (right panel). In the first lane are protein size markers, the second lane was loaded 2 μl tissue culture supernatant from HEK293 cells 4 days post transfection with plasmid DNA, and the third lane of the gel is 4 μl purified gE off the lectin affinity column.

### DETAILED DESCRIPTION

### VLPs of the Invention and Methods of Making VLPs

As used herein, the term “virus-like particle” (VLP) refers to a structure that in at least one attribute resembles a virus but which has been demonstrated to be non-infectious. Virus-like particles in accordance with the invention do not carry genetic information encoding for the proteins of the virus-like particles. In general, virus-like particles lack a viral genome and cannot replicate. In addition, virus-like particles can often be produced in large quantities by heterologous expression and can be easily purified. The term also refers to any subviral particle produced by the methods described below. This term includes protein aggregates which can be purified by any of the methods described below or known in the art.

As used herein, the term “antigenic formulation” or “antigenic composition” refers to a preparation which, when administered to a vertebrate, e.g. a mammal, will induce an immune response.

As used herein, the term “purified VLPs” refers to a preparation of VLPs of the invention that is at least 50%, 55% 60%, 65%, 70%, 75%, 80%, 85%, 90%, 95%, 96%, 97%, 98%, 99% or greater, free from other molecules (exclusive of solvent) in a mixture. For example, VLPs of the invention can be substantially free of other viruses, proteins, lipids, and carbohydrates associated with making VLPs of the invention. The term also encompasses VLPs which have been isolated from VLPs which have contaminating VZV genomic DNA or portions thereof.

As used herein, the term “chimeric VLP” refers to VLPs that contain proteins, or portions thereof, from at least two different infectious agents (heterologous proteins). Usually, one of the proteins is a derived from a virus that can drive the formation of VLPs from host cells (e.g. VZV gE) and the other protein is from a heterologous infectious agent.

The term “infectious agent” refers to microorganisms that cause an infection in a vertebrate. Infectious agents can be viruses, fungi, bacteria and/or parasites. A protein that may be expressed on the surface of VZV VLPs can be derived from viruses, fungi, bacteria and/or parasites. The proteins derived from viruses, fungi, bacteria and/or parasites can induce an immune response (cellular and/or humoral) in a vertebrate that which will prevent, treat, manage and/or ameliorate an infectious disease in said vertebrate.

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs.

As used herein, the term “effective amount” refers to an amount of VLPs necessary or sufficient to realize a desired biologic effect. An effective amount of the composition would be the amount that achieves a selected result, and such an amount could be determined as a matter of routine by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, the term “protective immunity” or “protective immune response” refers to immunity or eliciting an immune response against an infectious agent, which is exhibited by a vertebrate (e.g., a human), that prevents or ameliorates an infection or reduces at least one symptom thereof.

As used herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered.

The inventors have discovered that expressing VZV gE protein in cells induces VLP formation. Thus, the invention comprises VZV VLPs formed from the expression of VZV gE. The inventors have also developed novel VLPs from host cells that express gE and at least one additional VZV protein. In addition, these VLPs can also express antigenic proteins from other infectious agents. The invention also encompasses methods of making and administering an “antigenic formulation” comprised of the VZV VLPs of the invention. The invention also encompasses methods of making and administering a vaccine comprised of the VZV VLPs of the invention. This novel vaccine formulation overcomes some of the problems and concerns encountered with the currently available vaccine made from live attenuated virus.

The invention comprises a VZV VLP comprising VZV gE, wherein said VZV VLP does not comprise VZV nucleic acid or a yeast Ty protein. In another embodiment, VLPs of the invention consist essentially of gE protein. In another embodiment, said VZV VLP does not comprise VZV capsid proteins (e.g. ORF 20, ORF 40, ORF 41). In another embodiment, VLPs of the invention comprise at least one additional VZV protein incorporated into the VLP. In another embodiment, said additional VZV protein comprises gI (ORF 67) protein. In another embodiment, said additional VZV protein comprises gM (ORF 50) protein. In another embodiment, said additional VZV protein is gH. In another embodiment, said additional VZV protein is gB. In another embodiment, said additional VZV protein comprises at least one tegument protein. In another embodiment, said additional VZV protein comprises a combination of gI, gM, gH, gB or tegument proteins.

Another embodiment of the invention comprises chimeric VZV VLPs, which comprise a VZV gE protein and at least one protein from another infectious agent. In one embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

Another type of chimeric VLP of the invention also comprises a VLP comprising a VZV gE protein, at least one other protein from VZV, and at least one protein from another infectious agent. In one embodiment, said other protein from VZV is gI (ORF 67). In another embodiment, said other protein from VZV is gM (ORF 50). In another embodiment, said additional VZV protein is gH. In another embodiment, said additional VZV protein is gB. In another embodiment, said other protein from VZV is a tegument protein. In another embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

Non-limiting examples of viruses from which said infectious agent proteins can be derived from are the following: influenza (A and B, e.g. HA and/or NA), coronavirus (e.g. SARS), hepatitis viruses A, B, C, D & E3, human immunodeficiency virus (HIV), herpes viruses 1, 2, 6 & 7, cytomegalovirus, varicella zoster, papilloma virus, Epstein Barr virus, adenoviruses, bunya viruses (e.g. hanta virus), coxsakie viruses, picoma viruses, rotaviruses, rhinoviruses, rubella virus, mumps virus, measles virus, Rubella virus, polio virus (multiple types), adeno virus (multiple types), parainfluenza virus (multiple types), avian influenza (various types), shipping fever virus, Western and Eastern equine encephalomyelitis, Japanese encephalomyelitis, fowl pox, rabies virus, slow brain viruses, rous sarcoma virus, Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), Togaviridae (e.g., Rubivirus), Newcastle disease virus, West Nile fever virus, Tick borne encephalitis, yellow fever, chikungunya virus, respiratory syncytial virus, and dengue virus (all serotypes).

In another embodiment, the specific proteins from viruses may comprise: HA and/or NA from influenza virus (including avian), S protein from coronavirus, gp160, gp140 and/or gp41 from HIV, F or G proteins from respiratory syncytial virus, E and preM/M from yellow fever virus, Dengue (all serotypes) or any flavivirus. Also included are any protein from a virus that can induce an immune response (cellular and/or humoral) in a vertebrate that can prevent, treat, manage and/or ameliorate an infectious disease in said vertebrate.

Non-limiting examples of bacteria from which said infectious agent proteins can be derived from are the following: B. pertussis, Leptospira pomona, S. paratyphi A and B, C. diphtherias, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Campylobacter, Chlamydia, Coccidioides, Corynebacterium (e.g., Corynebacterium diptheriae), E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas. Treponema pollidum, Staphylococcus aureus, Pasteurella haemolytica, Corynebacterium diptheriae toxoid, Meningococcal polysaccharide, Bordetella pertusis, Streptococcus pneumoniae, Clostridium tetani toxoid, and Mycobacterium hovis.

Non-limiting examples of parasites from which said infectious agent proteins can be derived from are the following: leishmaniasis (Leishmania tropica mexicana, Leishmania tropica, Leishmania major, Leishmania aethiopica, Leishmania braziliensis, Leishmania donovani, Leishmania infantum, Leishmania chagasi), trypanosomiasis (Trypanosoma brucei gambiense, Trypanosoma brucei rhodesiense), toxoplasmosis (Toxoplasma gondii), schistosomiasis (Schistosoma haematobium, Schistosoma japonicum, Schistosoma mansoni, Schistosoma mekongi, Schistosoma intercalatum), malaria (Plasmodium virax, Plasmodium falciparium, Plasmodium malariae and Plasmodium ovale) Amebiasis (Entamoeba histolytica), Babesiosis (Babesiosis microti), Cryptosporidiosis (Cryptosporidium parvum), Dientamoebiasis (Dientamoeba fragilis), Giardiasis (Giardia lamblia), Helminthiasis and Trichomonas (Trichomonas vaginalis).

Non-limiting examples of fungi from which said infectious agent proteins can be derived are from the following: Absidia (e.g. Absidia corymbifera), Ajellomyces (e.g. Ajellomyces capsulatus, Ajellomyces dermatitidis), Arthroderma (e.g. Arthroderma benhamiae, Arthroderma Arthroderma gypseum, Arthroderma incurvatum, Arthroderma otae, Arthroderma vanbreuseghemii), Aspergillus (e.g. Aspergillus fumigatus, Aspergillus niger), Candida (e.g. Candida albicans, Candida albicans var. stellatoidea, Candida dublinensis, Candida glabrata, Candida guilliermondii (Pichia guilliermondii), Candida krusei (Issatschenkia orientalis), Candida parapsilosis, Candida pelliculosa (Pichia anomala), Candida tropicalis), Coccidioides (e.g. Coccidioides immitis), Cryptococcus (e.g. Cryptococcus neoformans (Filobasidiella neoformans), Histoplasma (e.g. Histoplasma capsulatum (Ajellomyces capsulatus), Microsporum (e.g. Microsporum canis (Arthroderma otae), Microsporum fulvum (Arthroderma fulvum), Microsporum gypseum, Genus Pichia (e.g. Pichia anomala, Pichia guilliermondii), Pneumocystis (e.g. Pneumocystis jirovecii), Cryptosporidium, Malassezia furfur, Paracoccidiodes. The above lists are meant to be illustrative and by no means are meant to limit the invention to those particular bacterial, viral, fungal or parasitic organisms.

The invention also encompasses variants of the said glycoproteins (or proteins) expressed on or in the VLPs of the invention (including said chimeras). The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating of gE, gI, gM, gH, gB or tegument proteins of VZV, etc. Thus, the invention also encompasses using known methods of protein engineering and recombinant DNA technology to improve or alter the characteristics of the proteins expressed on or in the VLPs of the invention. Various types of mutagenesis can be used to produce and/or isolate variant nucleic acids that encode for protein molecules and/or to further modify/mutate the proteins in or on the VLPs of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like.

Methods of cloning VZV proteins of the invention are known in the art. For example, the gene encoding a specific VZV protein can be isolated by RT-PCR from polyadenylated mRNA extracted from cells which had been infected with a VZV virus. The resulting product gene can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids.

Thus, the invention comprises nucleotides that encode proteins cloned into an expression vector that can be expressed in a cell that induces the formation of VLPs of the invention. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for the VZV gE (ORF 68) protein. In another embodiment, said vector comprises nucleotides that encode the VZV gE and at least one additional protein from an infectious agent. In another embodiment, said vector comprises nucleotides that encode the VZV gE protein and gI (ORF 67), gM (ORF 50), gH, gB and/or tegument VZV proteins. In another embodiment, the expression vector is a baculovirus vector.

In some embodiments of the invention proteins may comprise, mutations containing alterations which produce silent substitutions, additions, or deletions, but do not alter the properties or activities of the encoded protein or how the proteins are made. Nucleotide variants can be produced for a variety of reasons, e.g., to optimize codon expression for a particular host (change codons in the human mRNA to those preferred by insect cells such as Sf9 cells).

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the VZV viral proteins can be cloned. A person with skill in the art would understand that additional methods are available and are possible.

The invention also provides for constructs and/or vectors that comprise VZV nucleotides that encode for VZV proteins, including gE, gI, gM, gH, gB, tegument proteins or portions thereof. The vector may be, for example, a phage, plasmid, viral, or retroviral vector. The constructs and/or vectors that comprise VZV genes, including gE, gI, gM, gH, gB tegument proteins or portions thereof, should be operably linked to an appropriate promoter, such as the AcMNPV polyhedrin promoter (or other baculovirus), phage lambda PL promoter, the E. coil lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among preferred vectors are viral vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan.

Next, the recombinant constructs mentioned above could be used to transfect, infect, or transform and can express VZV proteins, including gE, gI, gM, gH, gB, tegument proteins, or portions thereof, into eukaryotic cells and/or prokaryotic cells. Thus, the invention provides for host cells which comprise a vector (or vectors) that contain nucleic acids which code for VZV proteins, including gE, gI, gM, gH, gB, tegument proteins, or portions thereof, and permit the expression of VZV genes, including gE, gI, gM, gH, gB, tegument proteins, or portions thereof, in said host cell under conditions which allow the formation of VLPs.

Among eukaryotic host cells are yeast, insect, amphibian, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. Lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

Vectors, e.g., vectors comprising polynucleotides of VZV gE, gI, gM, gH, gB, tegument proteins or portions thereof, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

In another embodiment, said vector and/or host cell comprise nucleotides that encode VZV protein gE, or portions thereof. In another embodiment, said vector and/or host cell consists essentially of nucleotides that encode VZV protein gE, or portions thereof. In a further embodiment, said vector and/or host cell comprise nucleotides that encode VZV proteins gE, gI, gM, gH, gB, tegument or portions thereof. The vectors and/or host cells described above contain VZV gE, gI, gM, gH, gB, tegument proteins, or portions thereof, and optionally any additional proteins from an infectious agent, and may contain additional cellular constituents such as cellular proteins, baculovirus proteins, lipids, carbohydrates etc., but do not contain Ty transposons or any protein encoded by a Ty transposon.

The invention also provides for methods of producing VLPs, said methods comprising expressing VZV genes including gE, gI, gM, gH, gB, tegument or portions thereof, and optionally any additional protein from an infectious agent under conditions that allow VLP formation. Depending on the expression system and host cell selected, the VLPs are produced by growing host cells transformed by an expression vector under conditions whereby the recombinant proteins are expressed and VLPs are formed. In one embodiment, the invention comprises a method of producing a VLP, comprising transfecting a vector encoding VZV gE protein into a suitable host cell and expressing said VZV gE protein under conditions that allow VLP formation. In another embodiment, said eukaryotic cell is selected from the group consisting of, yeast, insect, amphibian, avian or mammalian cells. The selection of the appropriate growth conditions is within the skill of one of ordinary skill in the art.

Methods to grow cells engineered to produce VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored.

The VLPs are then isolated using methods that preserve the integrity thereof, such as by gradient centrifugation, e.g., cesium chloride, sucrose and iodixanol, as well as standard purification techniques including, e.g., ion exchange and gel filtration chromatography. In one embodiment, the invention comprises purified VLPs of the invention. In another embodiment, said VLPs of the invention are at least 50%, 55% 60%, 65%, 70%, 75%, 80%, 85%, 90%, 95%, 96%, 97%, 98%, 99% or greater, free from other molecules (exclusive of solvent) present in a mixture. In another embodiment, said VLPs of the invention are substantially free of other viruses, proteins, lipids, and carbohydrates associated with making VLPs of the invention. The following is an example of how VLPs of the invention can be made, isolated and purified. Usually VLPs are produced from recombinant cell lines engineered to create VLPs when said cells are grown in cell culture (see above). A person of skill in the art would understand that there are additional methods that can be utilized to make and purify VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCell-420, JRH). Next, said cells are infected with recombinant baculovirus at the most efficient multiplicity of infection (e.g. from about 1 to about 3 plaque forming units per cell). Once infection has occurred, the VZV gE and/or other proteins self assemble into VLPs and are secreted from the cells approximately 24 to 72 hours post infection. Usually, infection is most efficient when the cells are in mid-log phase of growth (4-8×10⁶ cells/ml) and are at least about 90% viable.

VLPs of the invention can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μM filter cartridge or a similar device.

Next, VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl-105466 in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluted via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour. After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

### Pharmaceutical or Vaccine Formulations and Administration

The pharmaceutical compositions useful herein contain a VLP of the invention and a pharmaceutically acceptable carrier, including any suitable diluent or excipient, which includes any pharmaceutical agent that does not itself induce the production of an immune response harmful to the vertebrate receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic formulations for inducing a protective immune response in a vertebrate.

The invention encompasses an antigenic formulation comprising a VLP which comprises VZV gE protein, but does not include VZV nucleic acid or a yeast Ty protein. In one embodiment, said antigenic formulation comprises a VLP consisting essentially of gE protein. In another embodiment, said antigenic formulation comprises a VLP comprising at least one additional VZV protein incorporated into the VLP. In another embodiment, said additional VZV protein comprises gI (ORF 67) protein. In another embodiment, said additional VZV protein comprises gM (ORF 50) protein. In another embodiment, said additional VZV protein is gH. In another embodiment, said additional VZV protein is gB. In another embodiment, said additional VZV protein comprises a tegument protein. In another embodiment, said additional VZV protein comprises a combination of gI, gM, gH, gB or tegument proteins. In another embodiment, said VZV VLP does not comprise VZV capsid proteins (e.g. ORF 20, ORF 40, ORF 41).

The invention also encompasses an antigenic formulation comprising a chimeric VLP that comprises a VZV gE protein and at least one protein from another infectious agent. In one embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP. The invention also provides for an antigenic formulation comprising a purified chimeric VLP that comprises a VZV gE protein, at least one other protein from VZV, and at least one protein from another infectious agent. In one embodiment, said other protein from VZV is gI (ORF 67). In another embodiment, said other protein from VZV is gM (ORF 50). In another embodiment, said additional VZV protein is gH. In another embodiment, said additional VZV protein is gB. In another embodiment, said other protein from VZV is a tegument protein. In another embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The invention also encompasses a vaccine formulation comprising a VLP which comprises VZV gE protein, but does not include VZV nucleic acid or a yeast Ty protein. In one embodiment, said vaccine formulation comprises a VLP consisting essentially of gE protein. In another embodiment, said vaccine formulation comprises a VLP comprising at least one additional VZV protein incorporated into the VLP. In another embodiment, said additional VZV protein comprises gI (ORF 67) protein. In another embodiment, said additional VZV protein comprises gM (ORF 50) protein. In another embodiment, said additional VZV protein is gH. In another embodiment, said additional VZV protein is gB. In another embodiment, said additional VZV protein comprises a tegument protein. In another embodiment, said additional VZV protein comprises a combination of gI, gM, gH, gB or tegument proteins. In another embodiment, said VZV VLP does not comprise VZV capsid proteins (e.g. ORF 20, ORF 40, ORF 41).

The invention also encompasses a vaccine formulation comprising a chimeric VLP that comprises a VZV gE protein and at least one protein from another infectious agent. In one embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

The invention also provides for a vaccine formulation comprising a chimeric VLP that comprises a VZV gE protein, at least one other protein from VZV, and at least one protein from another infectious agent. In one embodiment, said other protein from VZV is gI (ORF 67). In another embodiment, said other protein from VZV is gM (ORF 50). In another embodiment, said additional VZV protein is gH. In another embodiment, said additional VZV protein is gB.

In another embodiment, said other protein from VZV is a tegument protein. In another embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

Said antigenic and vaccine formulations of the invention comprise VLPs of the invention as described above and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The pharmaceutical composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention provides that the VLP formulation be packaged in a hermetically sealed container such as an ampoule or sachette indicating the quantity of composition. In one embodiment, the VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the VLP composition is supplied in liquid form in a hermetically sealed container indicating the quantity and concentration of the VLP composition. Preferably, the liquid form of the VLP composition is supplied in a hermetically sealed container at least about 50 μg/ml, more preferably at least about 100 μg/ml, at least about 200 μg/ml, at least 500 μg/ml, or at least 1 μg/ml.

Generally, VZV VLPs of the invention are administered in an effective amount or quantity sufficient to stimulate an immune response against one or more strains of VZV. Preferably, administration of the VLP of the invention elicits immunity against VZV. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including VZV.

Thus, the invention also comprises a method of formulating a vaccine or antigenic composition that induces immunity to an infection or at least one symptom thereof to a mammal, comprising adding to said formulation an effective dose of a VZV VLP. In one embodiment, said infection is a VZV infection. An “effective dose” generally refers to that amount of VLPs of the invention sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provide a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

While stimulation of immunity with a single dose is preferred, additional dosages can be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections, e.g. VZV infection. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Methods of administering a composition comprising VLPs (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered orally, intradermally, intranasally, intramuscularly, intraperitoneally, intravenously, or subcutaneously. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising VLPs of the invention may induce an antibody or other immune response that is substantially higher than other routes of administration. In another embodiment, intranasal or other mucosal routes of administration of a composition comprising VLPs of the invention may induce an antibody or other immune response that will induce cross protection against other strains of VZV. Administration can be systemic or local.

Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, said VLPs of the invention can be administered as part of a combination therapy. For example, VLPs of the invention can be formulated with other immunogenic compositions, antivirals and/or antibiotics.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Said dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. VLPs of the invention, to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. The term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification Of the immune response can also mean decreasing or suppressing certain antigen-specific immune responses. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasome®, MHC antigens may also be used.

In one embodiment of the invention, the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

The VLPs of the invention can also be formulated with “immune stimulators.” The term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as VLPs of the invention, or can be administered separately. Either the protein or an expression vector encoding the protein can be administered to produce an immunostimulatory effect. Thus in one embodiment, the invention comprises antigenic and vaccine formulations comprising an adjuvant and/or an immune stimulator.

### Methods of Stimulating an Immune Response

The VLPs of the invention are useful for preparing compositions that stimulate an immune response that confers immunity or substantial immunity to infectious agents. Both mucosal and cellular immunity may contribute to immunity to infectious agents and disease. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. Protection of the respiratory tract is important in the case of VZV infection since, unlike other herpesviruses, VZV is transmissible through the respiratory system. The immune response induced by an infection protects against reinfection with the same virus or an antigenically similar viral strain.

VLPs of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates VZV infection or reduces at least one symptom thereof.

The invention encompasses a method of inducing protective immunity to an infection in a subject, comprising administering to the subject an antigenic formulation or vaccine comprising VZV-VLPs, wherein said VZV-VLPs comprise VZV gE protein, but does not include VZV nucleic acid or a yeast Ty protein. In one embodiment, said infection is caused by a virus. In another embodiment, said infection is caused by a fungus. In another embodiment, said infection is caused by a parasite. In another embodiment, said infection is caused by a bacterium. In another embodiment, said VZV-VLPs consist essentially of VZV gE protein. In another embodiment, said VZV-VLPs are derived from a recombinant expression system comprising a cloned VZV gE. In another embodiment, said VZV-VLPs comprise at least one additional VZV protein incorporated into the VLP. In another embodiment, said additional VZV protein comprises gI (ORF 67) protein. In another embodiment, said additional VZV protein comprises gM (ORF 50) protein. In another embodiment, said additional VZV protein is gH. In another embodiment, said additional VZV protein is gB. In another embodiment, said additional VZV protein comprises a tegument protein. In another embodiment, said additional VZV protein comprises a combination of gI, gM, gH, gB or tegument proteins.

Another embodiment of the invention comprises a method of inducing protective immunity to an infection in a subject, comprising administering to the subject an antigenic formulation or vaccine comprising VZV-VLPs, wherein said VZV-VLPs comprise chimeric VLPs that comprise a VZV gE protein and at least one protein from another infectious agent. In one embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

The invention also provides for a method of inducing protective immunity to an infection in a subject, comprising administering to the subject an antigenic formulation or vaccine comprising VZV-VLPs, wherein said VZV-VLPs comprise chimeric VLPs that comprise a VZV gE protein, at least one other protein from VZV, and at least one protein from another infectious agent. In one embodiment, said other protein from VZV is gI (ORF 67). In another embodiment, said other protein from VZV is gM (ORF 50). In another embodiment, said additional VZV protein is gH. In another embodiment, said additional VZV protein is gB. In another embodiment, said other protein from VZV is a tegument protein. In another embodiment, said protein from another infectious agent is a viral protein. In another embodiment, said protein from another infectious agent is a bacterial protein. In another embodiment, said protein from another infectious agent is a fungal protein. In another embodiment, said protein from another infectious agent is a protein from a parasite. In another embodiment, said protein from another infectious agent is expressed on the surface of the VLP.

As mentioned above, the VLPs of the invention prevent or reduce at least one symptom of VZV infection in a subject. Symptoms of the two diseases caused by VZV infection are well known in the art. Symptoms of chickenpox (varicella), produced by primary VZV infection, include fever, malaise, headache, abdominal pain, fatigue, anorexia, and skin lesions occurring predominantly on the scalp, face, and trunk. Shingles (herpes zoster), resulting from a reactivation of the latent VZV, is characterized by the following symptoms: a skin rash usually appearing unilaterally in a thoracic dermatome, acute neuritic pain, and hypersensitivity. Thus, the method of the invention comprises the prevention or reduction of at least one symptom associated with VZV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a VZV infection or additional symptoms, a reduced severity of VZV symptoms or a suitable assays (e.g. antibody titer and/or T-cell activation assay). The objective assessment comprises both animal and human assessments.

This invention is further illustrated by the following examples that should not be construed as limiting. The contents of all references, patents and published patent applications cited throughout this application, as well as the Figures, are incorporated herein by reference for all purposes.

### EXAMPLES

### Example 1

### Cells, Viruses, and Constructs

Spodoptera frugiperda Sf9 insect cells (ATCC CRL-1711) were maintained as suspension cultures in HyQ-SFX insect serum free medium (HyClone, Logan, Utah) at 28° C. A Bac-to-Bac baculovirus expression system (Invitrogen, Carlsbad, Calif.) was used with pFastBac1 transfer vectors in E. coli DHI OBac cells for the generation of recombinant baculovirus vectors expressing influenza genes.

VZV genes were based on GenBank sequence NC_{—}001348. Genes encoding the proteins (see below) were codon-optimized for high-level expression in Sf9 cells and synthesized at GeneArt (Regensburg, Germany): Synthetic genes were transferred into pFastBac1 downstream of the AcMNPV polyhedrin promoter, as described in detail previously for influenza virus (Pushko et al., 2005.)

Recombinant baculoviruses were generated by site-specific homologous recombination following transformation of transfer plasmids containing VZV genes of interest into E. coli DH10Bac competent cells, which contained the AcMNPV baculovirus genome (Invitrogen). The recombinant bacmid DNA was extracted from E. coli cells and transfected into the Sf9 cells using CellFectin (Invitrogen). The recombinant baculoviruses were recovered, plaque-purified, amplified, and the titers of recombinant baculovirus stocks were determined by agarose plaque assay using Sf9 cells.

The follow is the list of VZV protein and gene sequences cloned and expressed in rBV vectors and evaluated for the formation of VLPs:

ORF50 type 3 envelope glycoprotein M
(SEQ ID NO: 1)
MGTQKKGPRSEKVSPYDTTTPEVEALDHQMDTLNWRIWIIQVMMFTLG
AVMLLATLIAASSEYTGIPCFYAAVVDYELFNATLDGGVWSGNRGGYS
APVLFLEPHSVVAFTYYTALTAMAMAVYTLITAAIIHRETKNQRVRQS
SGVAWLVVDPTTLFWGLLSLWLLNAVVLLLAYKQIGVAATLYLGHFAT
SVIFTTYFCGRGKLDETNIKAVANLRQQSVFLYRLAGPTRAVFVNLMA
ALMAICILFVSLMLELVVANHLHTGLWSSVSVAMSTFSTLSVVYLIVS
ELILAHYIHVLIGPSLGTLVACATLGTAAHSYMDRLYDPISVQSPRLI
PTTRGTLACLAVFSVVMLLLRLMRAYVYHRQKRSRFYGAVRRVPERVR
GYIRKVKPAHRNSRRTNYPSQGYGYVYENDSTYETDREDELLYERSNS
GWE
ORF62 Transcriptional regultor ICP4
(SEQ ID NO: 2)
MDTPPMQRSTPQRAGSPDTLELMDLLDAAAAAAEHRARVVTSSQPDDL
LFGENGVMVGREHEIVSIPSVSGLQPEPRTEDVGEELTQDDYVCEDGQ
DLMGSPVIPLAEVFHTRFSEAGAREPTGADRSLETVSLGTKLARSPKP
PMNDGETGRGTTPPFPQAFSPVSPASPVGDAAGNDOREDQRSIPRQTT
RGNSPGLPSVVHRDRQTQSISGKKPGDEQAGHAHASGDGVVLQKTQRP
AQGKSPKKKTLKVKVPLPARKPGGPVPGPVEQLYHVLSDSVPAKGAKA
DLPFETDDTRPRKNDARGITPRVPGRSSGGKPRAFLALPGRSNAPDPI
EDDSPVEKKPKSREFVSSSSSSSSWGSSSEDEDDEPRRVSVGSETTGS
RSGREHAPSPSNSDDSDSNDGGSTKQNIQPGYRSISGPDPRIRKTKRL
AGEPGRQRQKSFSLPRSRTPIIPPVSGPLMMPDGSPWPGSAPLPSNRV
RFGPSGETREGHWEDEAVRAARARYEASTEPVPLYVPELGDPARQYRA
LINLIYCPDRDPIAWLQNPKLTGVNSALNQFYQKLLPPGRAGTAVTGS
VASPVPHVGEAMATGEALWALPHAAAAVAMSRRYDRAQKHFILQSLRR
AFASMAYPEATGSSPAARISRGHPSPTTPATQAPDPQPSAAARSLSVC
PPDDRLRTPRKRKSQPVESRSLLDKIRETPVADARVADDHVVSKAKRR
VSEPVTITSGPVVDPPAVITMPLDGPAPNGGFRRIPRGALHTPVPSDQ
ARKAYCTPETIARLVDDPLFPTAWRPALSFDPGALAEIAARRPGGGDR
RFGPPSGVEALRRRCAWMRQIPDPEDVRLLIIYDPLPGEDINGPLEST
LATDRGPSWSPSRGGLSVVLAALSNRLCLPSTHAWAGNWTGPPDVSAL
NARGVLLLSTRDLAFAGAVEYLGSRLASARRRLLVLDAVALERWPRDG
PALSQYHVYVRAPARPDAQAVVRWPDSAVTEGLARAVFASSRTFGPAS
FARIETAFANLYPGEQPLCLCRGGNVAYTVCTRAGPKTRVPLSPREYR
QYVLPGFDGCKDLARQSRGLGLGAADFVDEAAHSHRAANRWGLGAALR
PVFLPEGRRPGAAGPEAGDVPTWARVFCRHALLEPDPAAEPLVLPPVA
GRSVALYASADEARNALPPIPRVMWPPGFGAAETVLEGSDGTRFVFGH
HGGSERPSETQAGRQRRTADDREHALELDDWEVGCEDAWDSEEGGGDD
GDAPGSSEGVSIVSVAPGVLRDRRVGLRPAVKVELLSSSSSSEDEDDV
WGGRGGRSPPQSRG
(SEQ ID NO: 3)
ORF63 Regulatory protein ICP22
MECTSPATRGDSSESKPGASVDVNGKMEYGSAPGPLNGRDTSRGPGAF
CTPGWEIHPARLVEDINRVFLCIAQSSGRVTRDSRRLRRICLDFYLMG
RTRQRPTLACWEELLQLQPTQTQCLRATLMEVSHRPPRGEDGFIEAPN
VPLHRSALECDVSDDGGEDDSDDDGSTPSDVIEFRDSDAESSDGEDFI
VEEESEESTDSCEPDGVPGDCYRDGDGCNTRSPKRPQRAIERYAGAET
AEYTAAKALTALGEGGVDWKRRRHEAPRRHDIPPPHGV
Orf9 Tegument Protein VP22
(SEQ ID NO: 4)
MASSDGDRLCRSNAVRRKTTPSYSGQYRTARRSVVVGPPDDSDDSLGY
ITTVGADSPSPVYADLYFEHKNTTPRVHQPNDSSGSEDDFEDIDEVVA
AFREARLRHELVEDAVYENPLSVEKPSRSETKNAAVKPKLEDSPKRAP
PGAGAIASGRPISFSTAPKTATSSWCGPTPSYNKRVFCEAVRRVAAMQ
AQKAAEAAWNSNPPRNNAELDRLLTGAVIRITVNEGLNLIQAANEADL
GEGASVSKRGNNRKTGDLQGGMGNEPMYAQVRKPKSRTDTQTTGRITN
RSRARSASRTDTRK
Orf10 Tegument Protein VP16
(SEQ ID NO: 5)
MECNLGTEHPSTDTWNRSKTEQAVVDAFDESLFGDVASDIGFETSLYS
HAVKTAPSPPWVASPKILYQQLIRDLDFSEGPRLLSCLETWNEDLFSC
FPINEDLYSDMMVLSPDPDDVISTVSTKDHVEMFNLTTRGSVRLPSPP
KQPTGLPAYVQEVQDSFTVELRAREEAYTKLLVTYCKSIIRYLQGTAK
RTTIGLNIQNPDQKAYTQLRQSILLRYYREVASLARLLYLHLYLTVTR
EFSWRLYASQSAHPDVFAALKFTWTERRQFTCAFHPVLCNNGIVLLEG
KPLTASALREINYRRRELGLPLVRCGLVEENKSPIVQQPSFSVHLPRS
VGFLTHHIKRKLDAYAVKHPQEPRHVRADHPYAKVVENRNYGSSIEAM
ILAPPSPSEILPGDPPRPPTCGFLTR
Orf68E Type 1 env. glycoprotein E (gE)
(SEQ ID NO: 6)
MGTVNKPVVGVLMGFGIITGTLRITNPVRASVLRYDDFHTDEDKLDTN
SVYEPYYHSDHAESSWVNRGESSRKAYDHNSPYIWPRNDYDGFLENAH
EHHGVYNQGRGIDSGERLMQPTQMSAQEDLGDDTGIHVIPTLNGDDRH
KIVNVDQRQYGDVFKGDLNPKPQGQRLTEVSVEENHPFTLRAPIQRIY
GVRYTETWSFLPSLTCTGDAAPAIQHICLKHTTCFQDVVVDVDCAENT
KEDQLAEISYRFQGKKEADQPWIVVNTSTLFDELELDPPEIEPGVLKV
LRTEKQYLGVYIWNMRGSDGTSTYATFLVTWKGDEKTRNPTRAVTPQP
RGAEFHMWNYHSHVFSVGDTFSLAMHLQYKIHEAPFDLLLEWLYVPID
PTCQPMRLYSTCLYHPNAPQCLSHMNSGCTFTSPHLAQRVASTVYQNC
EHADNYTAYCLGISHMEPSFGLILHDGGTTLKFVDTPESESGLYVFVV
YFNGHVEAVAYTVVSTVDHFVNAIEERGFPPTAGQPPATTKPKEITPV
NPGTSPLLRYAAWTGGLAAVVLLCLVIFLICTAKRMRVKAYRVDKSPY
NQSMYYAGLPVDDFEDSESTDTEEEFGNAIGGSHGGSSYTVYIDKTR
Orf68TCM variant of gE; transmembrane (TM)
domain and COOH of gE replaced with influenza
A/Fujian TM domain and COOH (underlined)
(SEQ ID NO: 7)
MGTVNKPVVGVLMGFGIITGTLRITNPVRASVLRYDDFHTDEDKLDTN
SVYEPYYHSDHAESSWVNRGESSRKAYDHNSPYIWPRNDYDGFLENAH
EHHGVYNQGRGIDSGERLMQPTQMSAQEDLGDDTGIHVIPTLNGDDRH
KIVNVDQRQYGDVFKGDLNPKPQGQRLIEVSVEENHPFTLRAPIQRIY
GVRYTETWSFLPSLTCTGDAAPAIQHICLKHTTCFQDVVVDVDCAENT
KEDQLAEISYRFQGKKEADQPWIVVNTSTLFDELELDPPEIEPGVLKV
LRTEKQYLGVYIWNMRGSDGTSTYATFLVTWKGDEKTRNPTPAVTPQP
RGAEFHMWNYHSHVFSVGDTFSLAMHLQYKIHEAPFDLLLEWLYVPID
RTCQPMRLYSTCLYNPNAPQCLSHMNSGCTFTSPHLAQRVASTVYQNC
EHADNYTAYCLGISHMEPSFGLILHDGGTTLKFVDTPESLSGLYVFVV
YFNGHVEAVAYTVVSTVDHFVNAIEERGFPPTAGQPPATTKPKEITPV
NPGTSPLLRDWILWISFAISCFLLCVALLGFIMWACQKGNIRCNICI

Codon optimized VZV DNA sequences for insect cells are into baculovirus vectors and evaluated for assembly of VLPs.

ORF37 glycoprotein H (gH)
(SEQ ID NO: 8)
MFALVLAVVILPLWTTANKSYVTPTPATRSIGHMSALLREYSDRNMSL
KLEAFYPTGFDEELIKSLHWGNDRKHVFLVIVKVNPTTHEGDVGLVIF
PKYLLSPYHFKAEHRAPFPAGRFGFLSHPVTPDVSFFDSSFAPYLTTQ
HLVAFTTFPPNPLVWHLERAETAATAERPFGVSLLPARPTVPKNTILE
HKAHFATWDALARHTFFSAEAIITNSTLRIHVPLFGSVWPIRYWATGS
VLLTSDSGRVEVNIGVGFMSSLISLSSGPPIELIVVPHTVKLNAVTSD
TTWFQLNPPGPDPGPSYRVYLLGRGLDMNFSKHATVDICAYPEESLDY
RYHLSMAHTEALRMTTKADQHDINEESYYHIAARIATSIFALSEMGRT
TEYFLLDEIVDVQYQLKFLNYILMRIGAGAHPNTISGTSDLIFADPSQ
LHDELSLLFGQVKPANVDYFISYDEARDQLKTAYALSRGQDHVNALSL
ARRVIMSIYKGLLVKQNLNATERQALFFASMILLNFREGLENSSRVLD
GRTTLLLMTSMCTAAHATQAALNIQEGLAYLNPSKHMFTIPNVYSPCM
GSLRTDLTEEIHVMNLLSAIPTRPGLNEVLHTQLDESEIFDAAFKTMM
IFTTWTAKDLHILHTHVPEVFTCQDAAARNGEYVLILPAVQGHSYVIT
RNKPQRGLVYSLADVDVYNPISVVYLSRDTCVSEHGVIETVALPHPDN
LKECLYCGSVFLRYLTTGAIMDIIIIDSKDTERQLAAMGNSTIPPFNP
DMHGDDSKAVLLFPNGTVVTLLGFERRQAIRMSGQYLGASLGGAFLAV
VGFGIIGWMLCGNSRLREYNKIPLT
ORF67 glycoprotein I (gI)
(SEQ ID NO: 9)
MFLIQCLISAVIFYIQVTNALIFKGDHVSLQVNSSLTSILIPMQNDNY
TEIKGQLVFIGEQLPTGTNYSGTLELLYADTVAFCFRSVQVIRYDGCP
RIRTSAFISCRYKHSWHYGNSTDRISTEPDAGVMLKITKPGINDAGVY
VLLVRLDHSRSTDGFILGVNVYTAGSHHNIHGVIYTSPSLQNGYSTRA
LFQQARLCDLPATPKGSGTSLFQHMLDLRAGKSLEDNPWLHEDVVTTE
TKSVVKEGIENHVYPTDMSTLPEKSLNDPPENLLIIIPIVASVMILTA
MVIVIVISVKRRRIKKHPIYRPNTKTRRGIQNATPESDVMLEAAIAQL
ATIREESPPHSVVNPFVK
ORF31 glycoprotein B (gB)
(SEQ ID NO: 10)
MFVTAVVSVSPSSFYESLQVEPTQSEDITRSAHLGDGDEIREAIHKSQ
DAETKPTFYVCPPPTGSTIVRLEPTRTCPDYHLGKNFTEGIAVVYKEN
IAAYKFKATVYYKDVIVSTAWAGSSYTQITNRYADRVPIPVSEITDTI
DKFGKCSSKATYVRNNHKVEAFNEDKNPQDMPLIASKYNSVGSKAWHT
TNDTYMVAGTPGTYRTGTSVNCIIEEVEARSIFPYDSFGLSTGDIIYM
SPFFGLRDGAYREHSNYAMDRFHQFEGYRQRDLDTRALLEPAARNFLV
TPHLTVGWNWKPKRTEVCSLVKWREVEDVVRDEYAHNFRFTMKTLSTT
FISETNEFNLNQIHLSQCVKEEARAIINRIYTTRYNSSHVRTGDIQTY
LARGGFVVVFQPLLSNSLARLYLQELVRENTNHSPQKHPTRNTRSRRS
VPVELRANRTITTTSSVEFAMLQFTYDHIQEHVNEMLARISSSWCQLQ
NRERALWSGLFPINPSALASTILDQRVKARILGDVISVSNCPELGSDT
RIILQNSMRVSGSTTRCYSRPLISIVSLNGSGTVEGQLGTDNELIMSR
DLLEPCVANHKRYFLFGHHYVYYEDYRYVREIAVHDVGMISTYVDLNL
TLLKDREFMPLQVYTRDELRDTGLLDYSEIQRRNQMHSLRFYDIDKVV
QYDSGTAIMQGMAQFFQGLGTAGQAVGHVVLGATGALLSTVHGFTTFL
SNPFGALAVGLLVLAGLVAAFFAYRYVLKLKTSPMKALYPLTTKGLKQ
LPEGMDPFAEKPNATDTPIEEIGDSQNTEPSVNSGFDPDKFREAQEMI
KYMTLVSAAERQESKARKKNKTSALLTSRLTGLALRNRRGYSRVRTEN
VTGV

VZV gE gene sequences, codon-optimized and cloned into baculovirus expression vector.

Orf68 gE
(SEQ ID NO: 11)
ATGGGCACCGTGAACAAGCCCGTGGTGGGCGTGCTGATGGGTTTCGGT
ATCATCACCGGCACCCTGCGTATCACCAACCCCGTGCGTGCTTCCGTG
CTGCGTTACGACGACTTCCACACCGACGAGGACAAGCTGGACACCAAC
TCCGTGTACGAGCCCTACTACCACTCCGACCACGCTGAGTCCTCTTGG
GTGAACCGTGGCGAGTCCTCCCGTAAGGCTTACGACCACAACTCCCCC
TACATCTGGCCCCGTAACGACTACGACGGTTTCCTCGAGAACGCTCAC
GAGCACCACGGTGTCTACAACCAGGGTCGTGGTATCGACTCCGGCGAG
CGTCTGATGCAGCCCACCCAGATGTCCGCTCAGGAGGACCTGGGCGAC
GACACCGGTATCCACGTGATCCCCACCCTGAACGGTGACGACCGTCAC
AAGATCGTGAACGTGGACCAGCGCCAGTACGGTGACGTGTTCAAGGGT
GACCTGAACCCCAAGCCCCAGGGCCAGCGTCTGATCGAGGTGTCCGTG
GAGGAGAACCACCCCTTCACCCTGCGTGCTCCCATCCAGCGTATCTAC
GGTGTCCGTTACACCGAGACCTGGTCCTTCCTCCCCTCCCTGACCTGC
ACCGGTGACGCTGCTCCCGCTATCCAGCACATCTGCCTGAAGCACACC
ACCTGCTTCCAGGACGTGGTGGTGGACGTGGACTGCGCTGAGAACACC
AAGGAGGACCAGCTGGCTGAGATCTCCTACAGGTTCCAGGGCAAGAAG
GAGGCTGACCAGCCCTGGATCGTGGTGAACACCTCCACCCTGTTCGAC
GAGCTGGAGCTGGACCCCCCCGAGATCGAGCCCGGTGTCCTGAAGGTG
CTGCGTACCGAGAAGCAGTACCTGGGCGTGTACATCTGGAACATGCGT
GGTTCCGACGGCACCTCCACCTACGCTACCTTCCTCGTGACCTGGAAG
GGTGACGAAAAGACCCGTAACCCCACCCCCGCTGTGACCCCCCAGCCC
CGTGGTGCTGAATTCCATATGTGGAACTACCACTCTCACGTGTTCTCC
GTGGGTGACACCTTCTCCCTGGCTATGCACCTGCAGTACAAGATCCAC
GAGGCTCCCTTCGACCTGCTGCTCGAGTGGCTGTACGTGCCCATCGAC
CCCACCTGCCAGCCCATGCGCCTGTACTCCACCTGCCTGTACCACCCC
AACGCTCCCCAGTGCCTGTCCCACATGAACTCCGGTTGCACCTTCACC
TCCCCCCACCTGGCCCAGCGTGTGGCTTCCACCGTGTACCAGAACTGC
GAGCACGCTGAcAACTACACCGCTTACTGCCTGGGTATCAGcCACATG
GAGCCTTCCTTCGGTCTGATCCTGCACGACGGTGGCACCACCCTGAAG
TTCGTGGACACCCCCGAGTCCCTGTCCGGTCTGTACGTGTTCGTGGTG
TACTTCAACGGTCACGTGGAGGCTGTCGCTTACACCGTGGTGTCCACC
GTGGACCACTTCGTGAACGCTATCGAGGAGCGTGGTTTCCCCCCCACC
GCTGGCCAGCCCCCTGCTACCACCAAGCCCAAGGAGATCACCCCCGTC
AACCCCGGCACCTCCCCTCTGCTGCGCTACGCTGCTTGGACCGGTGGT
CTGGCTGCTGTGGTGCTGCTGTGCCTGGTGATCTTCCTGATCTGCACC
GCTAAGAGGATGCGTGTGAAGGCTTACCGTGTGGACAAGTCCCCTTAC
AACCAGTCCATGTACTACGCTGGTCTGCCCGTCGACGACTTCGAGGAC
TCCGAGTCCACCGACACCGAGGAGGAGTTCGGTAACGCTATCGGTGGT
TCCCACGGTGGTTCCTCCTACACCGTGTACATCGACAAGACCCGCTAA
Codon-optimized sequence for Orf68TCM, start
and stop codons underlined
(SEQ ID NO: 12)
ATGGGCACCGTGAACAAGCCCGTGGTGGGCGTGCTGATGGGTTTCGGT
ATCATCACCGGCACCCTGCGTATCACCAACCCCGTGCGTGCTTCCGTG
CTGCGTTACGACGACTTCCACACCGACGAGGACAAGCTGGACACCAAC
TCCGTGTACGAGCCCTACTACCACTCCGACCACGCTGAGTCCTCTTGG
GTGAACCGTGGCGAGTCCTCCCGTAAGGCTTACGACCACAACTCCCCC
TACATCTGGCCCCGTAACGACTACGACGGTTTCCTCGAGAACGCTCAC
GAGCACCACGGTGTCTACAACCAGGGTCGTGGTATCGACTCCGGCGAG
CGTCTGATGCAGCCCACCCAGATGTCCGCTCAGGAGGACCTGGGCGAC
GACACCGGTATCCACGTGATCCCCACCCTGAACGGTGACGACCGTCAC
AAGATCGTGAACGTGGACCAGCGCCAGTACGGTGACGTGTTCAAGGGT
GACCTGAACCCCAAGCCCCAGGGCCAGCGTCTGATCGAGGTGTCCGTG
GAGGAGAACCACCCCTTCACCCTGCGTGCTCCCATCCAGCGTATCTAC
GGTGTCCGTTACACCGAGACCTGGTCCTTCCTCCCCTCCCTGACCTGC
ACCGGTGACGCTGCTCCCGCTATCCAGCACATCTGCCTGAAGCACACC
ACCTGCTTCCAGGACGTGGTGGTGGACGTGGACTGCGCTGAGAACACC
AAGGAGGACCAGCTGGCTGAGATCTCCTACAGGTTCCAGGGCAAGAAG
GAGGCTGACCAGCCCTGGATCGTGGTGAACACCTCCACCCTGTTCGAC
GAGCTGGAGCTGGACCCCCCCGAGATCGAGCCCGGTGTCCTGAAGGTG
CTGCGTACCGAGAAGCAGTACCTGGGCGTGTACATCTGGAACATGCGT
GGTTCCGACGGCACCTCCACCTACGCTACCTTCCTCGTGACCTGGAAG
GGTGACGAAAAGACCCGTAACCCCACCCCCGCTGTGACCCCCCAGCCC
CGTGGTGCTGAATTCCATATGTGGAACTACCACTCTCACGTGTTCTCC
GTGGGTGACACCTTCTCCCTGGCTATGCACCTGCAGTACAAGATCCAC
GAGGCTCCCTTCGACCTGCTGCTCGAGTGGCTGTACGTGCCCATCGAC
CCCACCTGCCAGCCCATGCGCCTGTACTCCACCTGCCTGTACCACCCC
AACGCTCCCCAGTGCCTGTCCCACATGAACTCCGGTTGCACCTTCACC
TCCCCCCACCTGGCCCAGCGTGTGGCTTCCACCGTGTACCAGAACTGC
GAGCACGCTGACAACTACACCGCTTACTGCCTGGGTATCAGCCACATG
GAGCCTTCCTTCGGTCTGATCCTGCACGACGGTGGCACCACCCTGAAG
TTCGTGGACACCCCCGAGTCCCTGTCCGGTCTGTACGTGTTCGTGGTG
TACTTCAACGGTCACGTGGAGGCTGTCGCTTACACCGTGGTGTCCACC
GTGGACCACTTCGTGAACGCTATCGAGGAGCGTGGTTTCCCCCCCACC
GCTGGCCAGCCCCCTGCTACCACCAAGCCCAAGGAGATCACCCCCGTC
AACCCCGGCACCTCCCCTCTGCTGCGCGACTGGATCTTGTGGATCTCC
TTCGCTATCTCCTGCTTCCTGCTGTGCGTGGCTCTGCTGGGTTTCATC
ATGTGGGCTTGCCAGAAGGGTAACATCCGTTGCAACATCTGCATCTAA

Alignment of ORF62 protein from GenBank NC_{—}001348 (VZV Dumas Strain) to ORF62 Protein from GenBank AB097933 (VZV Oka parental strain)

Query 1—Oka parental strain

Query 2—Dumas strain

| Identities = (99%) | | | |
|---|---|---|---|
| Query | 1 | MDTPPMQRSTPQRAGSPDTLELMDLLIMAAAAAEHRARVVTSSQPDDLLFGENGVMVGRE | 60 |
| | | MDTPPMQRSTPQRAGSPDTLELMDLLDAAAAAAEHRARVVTSSQPDDLLFGENGVMVGRE | |
| Sbjct | 1 | MDTPPMQRSTPQRAGSPDTLELMDLLDAAAAAAEHRARVVTSSQPDDLLFGENGVMVGRE | 60 |
| Query | 61 | HEIVSIPSVSGLQPEPRTEDVGEELTQDDYVCEDGQDLMGSPVIPLAEVFHTRFSEAGAR | 120 |
| | | HEIVSIPSVSGLQPEPRTEDVGEELTQDDYVCEDGQDLMGSPVIPLAEVFHTRFSEAGAR | |
| Sbjct | 61 | HEIVSIPSVSGLQPEPRTEDVGEELTQDDYVCEDGQDLMGSPVIPLAEVFHTRFSEAGAR | 120 |
| Query | 121 | EPTGADRSLETVSLGTKLARSPKPPMNDGETGRGTTPPFPQAFSPVSPASPVGDAAGNDQ | 180 |
| | | EPTGADRSLETVSLGTKLARSPKPPMNDGETGRGTTPPFPQAFSPVSPASPVGDAAGNDQ | |
| Sbjct | 121 | EPTGADRSLETVSLGTKLARSPKPPMNDGETGRGTTPPFPQAFSPVSPASPVGDAAGNDQ | 180 |
| Query | 181 | REDQRSIPRQTTRGNSPGLPSVVHRDRQTQSISGKKPGDEQAGHAHASGDGVVLQKTQRP | 240 |
| | | REDQRSIPRQTTRGNSPGLPSVVHRDRQTQSISGKKPGDEQAGHAHASGDGVVLQKTQRP | |
| Sbjct | 181 | REDQRSIPRQTTRGNSPGLPSVVHRDRQTQSISGKKPGDEQAGHAHASGDGVVLQKTQRP | 240 |
| Query | 241 | AQGKSPKKKTLKVKVPLPARKPGGPVPGPVEQLYHVLSDSVPAKGAKADLPFETDDTRPR | 300 |
| | | AQGKSPKKKTLKVKVPLPARKPGGPVPGPVEQLYHVLSDSVPAKGAKADLPFETDDTRPR | |
| Sbjct | 241 | AQGKSPKKKTLKVKVPLPARKPGGPVPGPVEQLYHVLSDSVPAKGAKADLPFETDDTRPR | 300 |
| Query | 301 | KHDARGITPRVPGRSSGGKPRAFLALPGRSHAPDPIEDDSPVEKKPKSREFVSSSSSSSS | 360 |
| | | KHDARGITPRVPGRSSGGKPRAFLALPGRSHAPDPIEDDSPVEKKPKSREFVSSSSSSSS | |
| Sbjct | 301 | KHDARGITPRVPGRSSGGKPRAFLALPGRSHAPDPIEDDSPVEKKPKSREFVSSSSSSSS | 360 |
| Query | 361 | WGSSSEDEDDEPRRVSVGSETTGSRSGREHAPSPSNSDDSDSNDGGSTKQNIQPGYRSIS | 420 |
| | | WGSSSEDEDDEPRRVSVGSETTGSRSGREHAPSPSNSDDSDSNDGGSTKQNIQPGYRSIS | |
| Sbjct | 361 | WGSSSEDEDDEPRRVSVGSETTGSRSGREHAPSPSNSDDSDSNDGGSTKQNIQPGYRSIS | 420 |
| Query | 421 | GPDPRIRKTKRLAGEPGRQRQKSFSLPRSRTPIIPPVSGPLMMPOGSPWPGSAPLPSNRV | 480 |
| | | GPDPRIRKTKRLAGEPGRQRQKSFSLPRSRTPIIPPVSGPLMMPDGSPWPGSAPLPSNRV | |
| Sbjct | 421 | GPDPRIRKTKRLAGEPGRQRQKSFSLPRSRTPIIPPVSGPLMMPDGSPWPGSAPLPSNRV | 480 |
| Query | 481 | RFGPSGETREGHWEDEAVRAARARYEASTEPVPLYVPELGDPARQYRALINLIYCPDRDP | 540 |
| | | RFGPSGETREGHWEDEAVRAARARYEASTEPVPLYVPELGDPARQYRALINLIYCPDRDP | |
| Sbjct | 481 | RFGPSGETREGHWEDEAVRAARARYEASTEPVPLYVPELGDPARQYRALINLIYCPDRDP | 540 |
| Query | 541 | IAWLQNPKLTGVNSALNQFYQKLLPPGRAGTAVTGSVASPVPHVGEAMATGEALWALPHA | 600 |
| | | IAWLQNPKLTGVNSALNQFYQKLLPPGRAGTAVTGSVASPVPHVGEAMATGEALWALPHA | |
| Sbjct | 541 | IAWLQNPKLTGVNSALNQFYQKLLPPGRAGTAVTGSVASPVPHVGEAMATGEALWALPHA | 600 |
| Query | 601 | AAAVAMSRRYDRAQKHFILQSLRRAFASMAYPEATGSSPAARISRGHPSPTTPATQ [symbol] PDP | 660 |
| | | AAAVAMSRRYDRAQKHFILQSLRRAFASMAYPEATGSSPAARISRGHPSPTTPATQ PDP | |
| Sbjct | 601 | AAAVAMSRRYDRAQKHFILQSLARAFASMAYPEATGSSPAARISRGHPSPTTPATQ [symbol] PDP | 660 |
| Query | 661 | QPSAAARSLSVCPPDDRLRTPRKRKSQPVESRSLLDKIRETPVADARVADDHVVSKAKRR | 720 |
| | | QPSAAARSLSVCPPDDRLRTPRKRKSQPVESRSLLDKIRETPVADARVADDHVVSKAKRR | |
| Sbjct | 661 | QPSAAARSLSVCPPDDRLRTPRKRKSQPVESRSLLDKIRETPVADARVADDHVVSKAKRR | 720 |
| Query | 721 | VSEPVTITSGPVVDPPAVITMPLDGPAPNGGFRRIPRGALHTPVPSDQARKAYCTPETIA | 780 |
| | | VSEPVTITSGPVVDPPAVITMPLDGPAPNGGFRRIPRGALHTPVPSDQARKAYCTPETIA | |
| Sbjct | 721 | VSEPVTITSGPVVDPPAVITMPLDGPAPNGGFRRIPRGALHTPVPSDQARKAYCTPETIA | 780 |
| Query | 781 | RLVDDPLFPTAWRPALSFDPGALAEIAARRPGGGDRRFGPPSGVEALRRRCAWMRQIPDP | 840 |
| | | RLVDDPLFPTAWRPALSFDPGALAEIAARRPGGGDRRFGPPSGVEALRRRCAWMRQIPDP | |
| Sbjct | 781 | RLVDDPLFPTAWRPALSFDPGALAEIAARRPGGGDRRFGPPSGVEALRRRCAWMRQIPDP | 840 |
| Query | 841 | EDVRLLIIYDPLPGEDINGPLESTLATDPGPSWSPSRGGLSVVLAALSNRLCLPSTHAWA | 900 |
| | | EDVRLLIIYDPLPGEDINGPLESTLATDPGPSWSPSRGGLSVVLAALSNRLCLPSTHAWA | |
| Sbjct | 841 | EDVRLLIIYDPLPGEDINGPLESTLATDPGPSWSPSRGGLSVVLAALSNRLCLPSTHAWA | 900 |
| Query | 901 | GNWTGPPDVSALNARGVLLLSTRDLAFAGAVEYLGSRLASARRRLLVLDAVALERWPRDG | 960 |
| | | GNWTGPPDVSALNARGVLLLSTRDLAFAGAVEYLGSRLASARRRLLVLDAVALERWPRDG | |
| Sbjct | 901 | GNWTGPPDVSALNARGVLLLSTRDLAFAGAVEYLGSRLASARRRLLVLDAVALERWPRDG | 960 |
| Query | 961 | PALSQYHVYVRAPARPDAQAVVRWPDSAVTEGLARAVFASSRTFGPASFARIETAFANLY | 1020 |
| | | PALSQYHVYVRAPARPDAQAVVRWPDSAVTEGLARAVFASSRTFGPASFARIETAFANLY | |
| Sbjct | 961 | PALSQYHVYVRAPARPDAQAVVRWPDSAVTEGLARAVFASSRTFGPASFARIETAFANLY | 1020 |
| Query | 1021 | PGEQPLCLCRGGNVAYTVCTRAGPKTRVPLSPREYRQYVLPGFDGCKDLARQSRGLGLGA | 1080 |
| | | PGEQPLCLCRGGNVAYTVCTRAGPKTRVPLSPREYRQYVLPGFDGCKDLARQSRGLGLGA | |
| Sbjct | 1021 | PGEQPLCLCRGGNVAYTVCTRAGPKTRVPLSPREYRQYVLPGFDGCKDLARQSRGLGLGA | 1080 |
| Query | 1081 | ADFVDEAAHSHRAANRWGLGAALRPVFLPEGRRPGAAGPEAGDVPTWARVFCRHALLEPD | 1140 |
| | | ADFVDEAAHSHRAANRWGLGAALRPVFLPEGRRPGAAGPEAGDVPTWARVFCRHALLEPD | |
| Sbjct | 1081 | ADFVDEAAHSHRAANRWGLGAALRPVFLPEGRRPGAAGPEAGDVPTWARVFCRHALLEPD | 1140 |
| Query | 1141 | PAAEPLVLPPVAGRSVALYASADEARNALPPIPRVMWPPGFGAAETVLEGSDGTRFVFGH | 1200 |
| | | PAAEPLVLPPVAGRSVALYASADEARNALPPIPRVMWPPGFGAAETVLEGSDGTRFVFGH | |
| Sbjct | 1141 | PAAEPLVLPPVAGRSVALYASADEARNALPPIPRVMWPPGFGAAETVLEGSDGTRFVFGH | 1200 |
| Query | 1201 | HGGSERP [symbol] ETQAGRQRRTADDREHALE [symbol] DDWEVGCEDAWDSEEGGGDDGDAPGSSFGVSI | 1260 |
| | | HGGSERP+ETQAGRQRRTADDREHALE DDWEVGCEDAWDSEEGGGDDGDAPGSSFGVSI | |
| Sbjct | 1201 | HGGSERP [symbol] ETQAGRQRRTADDREHALE [symbol] DDWEVGCEDAWDSEEGGGDDGDAPGSSFGVSI | 1260 |

| | | |
|---|---|---|
| Query | 1261 | VSVAPGVLRDRRVGLRPAVKVELLSSSSSSEDEDDVWGGRGGRSPPQSRG (SEQ ID NO: 13) |
| | | VSVAPGVLRDRRVGLRPAVKVELLSSSSSSEDEDDVWGGRGGRSPPQSRG (SEQ ID NO: 14) |
| Sbjct | 1261 | VSVAPGVLRDRRVGLRPAVKVELLSSSSSSEDEDDVWGGRGGRSPPQSRG (SEQ ID NO: 15) |

### Example 2

### Expression of VZV gE Protein Alone Forms VLPs

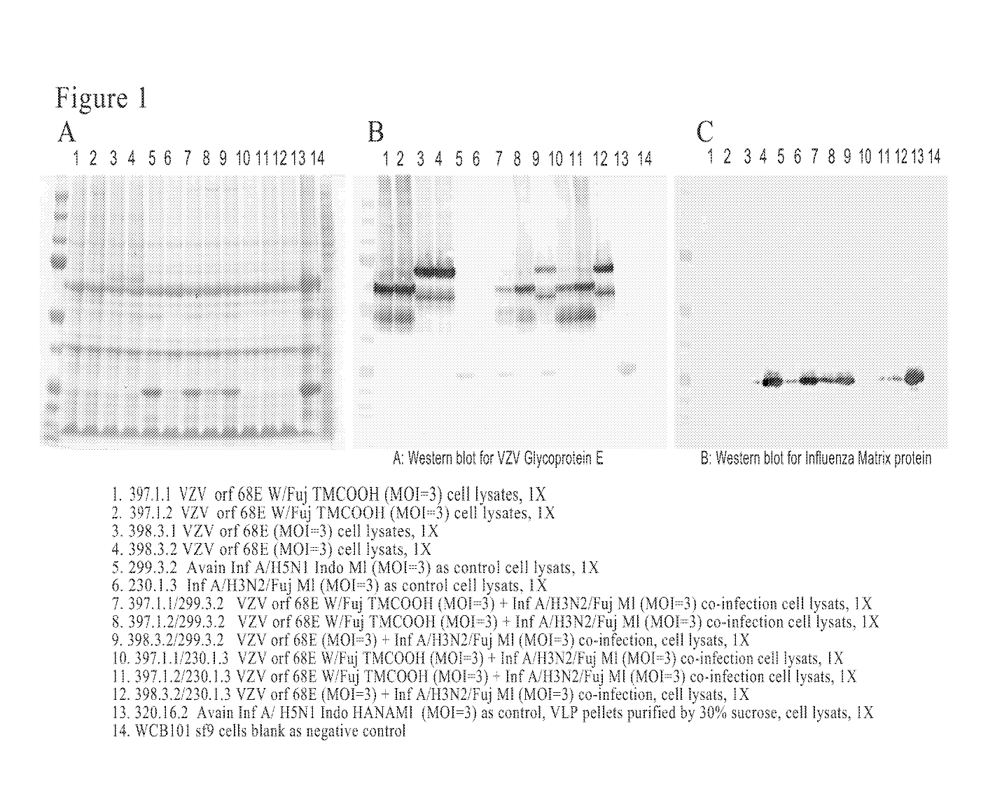

A baculovirus construct containing only VZV gE was expressed in SF9 cells and analyzed according to the above procedures. Particles were purified from Sf9 cells infected with a BV-VZV gE vector through the 20%-60% sucrose density gradient step using the process described above. Gels and Western blots confirmed that VZV gE was recovered in the particle fraction of the sucrose gradient. The samples were run on a SDS gel (FIG. 1A) and a western blot of the isolated supernatant was probed for VZV gE (FIG. 1B) or Influenza matrix protein (FIG. 1C). As shown in lanes 2 and 3 of FIG. 1B, expression of VZV gE protein alone lead to the formation of VZV-VLPs.

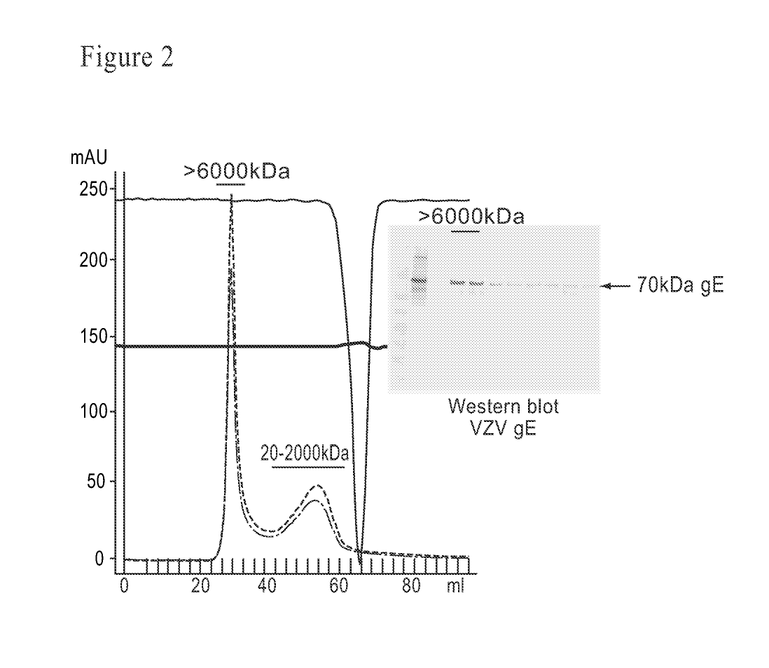

In addition, an analysis of gradient purified particles analyzed by size fractionation on a Sephacryl S-400 gel permeation chromatography column was performed. The majority of the gE protein is >6,000 kDa consistent with this being a VLP (FIG. 2). Thus, expression of VZV gE alone is sufficient to form virus like particles. In addition, as a control, an influenza M1 was expressed alone or with chimeric gE (gE fused to the transmembrane and cytoplasmic of influenza HA). These controls show that there was formation of VLPs made from expression of influenza M1 and the expression of M1 with chimeric VZV gE protein.

### Example 3

### Expression of IE62

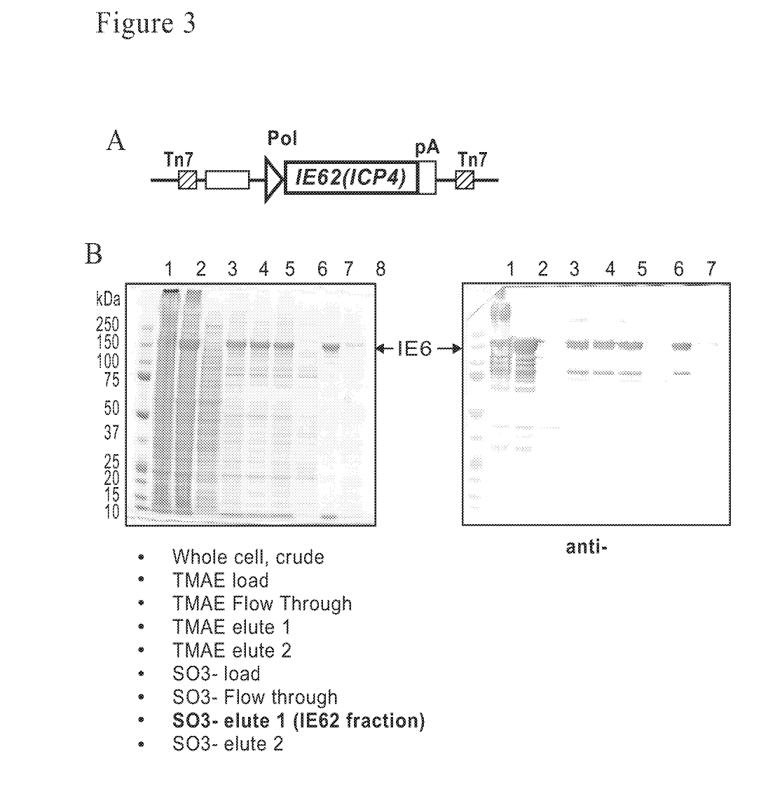

E62 is the major tegament protein of VZV. Immunization induces specific antibodies and cell mediated immunity (CMI) which protects guinea pigs when challenged with VZV. Described is a full length VZV IE62 gene cloned into a baculovirus expression vector (FIG. 3A), recombinant IE62 produced in Sf9 insect cells, and a non-denaturing process to extract and purify intracellular IE62.

Methods. A baculovirus was engineered to express a full length, codon optimized gene of IE62 from the Oka strain of VZV. The gene was synthesized (GeneArt, Germany) and cloned into a pFastBac1 vector under the control of the baculovirus polyhedrin promoter (Invitrogen). This gene was transferred to an AcMNPV baculovirus Bacmid (Invitrogen), the Bacmid DNA used to transfect Sf9 insect cells. The resulting recombinant baculovirus was plaque-purified and virus stock prepared in Sf9 cells.

Orf IE62 ICP4 full length.
(SEQ ID NO: 16)
ATGGACACCCCCCCCATGCAGCGTTCCACCCCCCAGCGTGCTGGTTCC
CCCGACACCCTCGAGCTGATGGACCTGCTGGACGCTGCTGCTGCCGCT
GCCGAGCACCGTGCTCGTGTGGTGACCTCCTCCCAGCCCGACGACCTG
CTGTTCGGCGAGAACGGTGTCATGGTCGGTCGTGAGCACGAGATCGTG
TCCATCCCTTCCGTGTCCGGTCTGCAGCCCGAGCCCCGTACCGAGGAC
GTGGGCGAAGAGCTGACCCAGGACGACTACGTGTGCGAGGACGGCCAG
GACCTGATGGGTTCCCCCGTGATCCCCCTGGCTGAGGTGTTCCACACC
CGTTTCTCCGAGGCTGGTGCTCGTGAGCCCACCGGTGCTGACCGTTCC
CTCGAGACCGTGTCCCTGGGCACCAAGCTGGCTCGTTCCCCCAAGCCC
CCCATGAACGACGGCGAGACCGGTCGTGGCACCACCCCCCCCTTCCCT
CAGGCTTTCTCCCCTGTGTCCCCCGCTTCCCCCGTGGGTGACGCTGCT
GGTAACGACCAGCGTGAGGACCAGCGTTCCATCCCTCGTCAGACCACC
CGTGGTAACTCCCCCGGTCTGCCCTCCGTGGTGCACCGTGACCGTCAG
ACCCAGTCCATCTCCGGCAAGAAGCCCGGCGACGAGCAGGCTGGTCAC
GCTCACGCTTCCGGTGACGGTGTTGTGCTGCAAAAAACCCAACGTCCC
GCCCAGGGAAAGTCTCCCAAGAAGAAAACCCTGAAGGTCAAGGTGCCC
CTGCCCGCTCGTAAGCCCGGTGGTCCCGTGCCCGGTCCCGTGGAGCAG
CTGTACCACGTGCTGTCCGACTCCGTGCCCGCTAAGGGTGCTAAGGCT
GACCTGCCTTTCGAGACCGACGACACCCGTCCCCGTAAGCATGACGCT
AGGGGCATCACTCCTCGTGTGCCCGGTCGTTCCTCCGGTGGCAAGCCC
CGTGCTTTCCTGGCTCTGCCTGGTCGTTCCCACGCTCCCGACCCCATC
GAGGACGACTCCCCCGTGGAGAAGAAGCCCAAGTCCCGCGAGTTCGTG
TCCTCCTCCTCCAGCTCCTCCTCCTGGGGTTCCAGCTCCGAGGACGAG
GACGACGAGCCCCGTCGTGTGTCCGTGGGTTCCGAGACCACCGGTTCC
CGTTCCGGTCGCGAGCACGCCCCCTCCCCATCCAACTCTGACGACTCC
GACTCCAACGACGGTGGTTCCACCAAGCAGAACATCCAGCCCGGCTAC
CGTTCCATTTCTGGTCCCGACCCCCGTATCCGTAAGACCAAGCGTCTG
GCTGGCGAACCAGGCCGTCAGCGTCAGAAGTCCTTCTCCCTGCCCCGT
TCCCGTACCCCTATCATCCCTCCTGTCTCCGGCCCTCTGATGATGCCC
GACGGTTCCCCCTGGCCCGGTTCCGCTCCCCTGCCCTCCAACCGTGTG
CGTTTCGGTCCCTCCGGCGAGACCCGTGAGGGCCACTGGGAGGACGAG
GCTGTGCGTGCTGCTCGTGCTCGTTACGAGGCTTCCACCGAGCCCGTG
CCCCTGTACGTGCCCGAACTGGGTGACCCTGCCCGTCAGTACCGTGCT
CTGATCAACCTGATCTACTGCCCCGACCGTGACCCCATCGCTTGGCTG
CAGAACCCCAAGCTGACCGGTGTCAACTCCGCTCTGAACCAGTTCTAC
CAGAAGCTGCTGCCCCCTGGTCGTGCTGGCACCGCTGTGACCGGTTCC
GTGGCTTCCCCTGTGCCCCACGTGGGAGAGGCTATGGCTACCGGCGAG
GCTCTGTGGGCTCTGCCTCACGCTGCCGCCGCTGTGGCTATGTCCCGT
CGTTACGACCGTGCTCAGAAGCACTTCATCCTGCAGTCCCTGCGTCGT
GCTTTCGCTTCCATGGCTTACCCCGAGGCTACCGGTTCCTCCCCCGCT
GCTCGTATCTCCCGTGGTCACCCCTCCCCCACCACCCCCGCTACCCAG
GCTCCAGACCCCCAACCCTCTGCTGCTGCTCGTTCCCTGTCCGTGTGC
CCCCCTGACGACCGTCTGCGTACCCCCCGTAAGCGCAAGTCCCAGCCC
GTGGAGTCCCGTTCCCTGCTGGACAAGATCCGTGAGACCCCAGTGGCT
GACGCTCGCGTGGCTGACGACCACGTCGTGTCCAAGGCTAAGAGGCGC
GTGTCCGAGCCTGTGACCATCACCTCCGGTCCTGTGGTGGACCCCCCT
GCTGTGATCACCATGCCCCTGGACGGTCCCGCTCCCAACGGTGGTTTC
CGTCGTATCCCTCGTGGTGCTCTGCACACCCCCGTGCCCTCCGACCAG
GCTCGTAAGGCTTACTGCACCCCCGAGACCATCGCTCGTCTGGTGGAC
GACCCCCTGTTCCCCACCGCTTGGCGTCCTGCTCTGTCCTTCGACCCC
GGTGCTCTGGCTGAGATCGCTGCTCGCCGTCCCGGTGGCGGTGATCGT
CGCTTCGGTCCTCCCTCCGGTGTCGAGGCTCTGCGTCGTCGTTGCGCT
TGGATGCGTCAGATCCCCGACCCTGAGGACGTGCGCCTGCTGATCATC
TACGACCCTCTGCCCGGCGAGGACATCAACGGTCCTCTCGAGTCCACC
CTGGCTACCGACCCCGGTCCCTCCTGGTCCCCCTCCCGTGGTGGTCTG
TCCGTGGTGCTGGCTGCCCTGTCCAACCGTCTGTGCCTGCCTTCCACC
CACGCTTGGGCTGGTAACTGGACCGGTCCCCCCGACGTGTCCGCCCTG
AACGCTCGCGGTGTCTTGCTCCTGTCCACCCGTGATCTGGCTTTCGCT
GGTGCTGTGGAGTACCTGGGTTCCCGTCTGGCTTCCGCTCGTCGTCGT
CTGCTGGTCCTGGACGCTGTGGCTCTCGAGCGTTGGCCCCGTGACGGT
CCAGCCCTGTCTCAATACCACGTGTACGTGCGCGCTCCCGCTCGTCCC
GACGCTCAGGCTGTGGTGCGCTGGCCCGACTCCGCTGTCACCGAGGGT
CTGGCTCGTGCTGTGTTCGCTTCCTCCCGTACCTTCGGTCCCGCTTCC
TTCGCTCGTATCGAGACCGCTTTCGCTAACCTGTACCCCGGCGAGCAG
CCCCTGTGCCTGTGCCGTGGTGGTAACGTGGCTTACACCGTGTGCACC
CGTGCTGGTCCCAAGACCCGTGTGCCTCTGTCCCCCCGTGAGTACCGC
CAGTACGTGCTGCCCGGTTTCGACGGTTGCAAGGACCTGGCTCGTCAG
TCCCGCGGTCTGGGTCTGGGTGCTGCTGACTTCGTCGACGAGGCTGCT
CACTCCCACCGTGCTGCTAACCGTTGGGGCCTGGGCGCTGCTCTGCGT
CCCGTGTTCCTGCCCGAGGGTCGTCGTCCTGGTGCTGCTGGTCCCGAG
GCTGGCGACGTGCCCACCTGGGCTCGTGTGTTCTGCCGTCACGCTCTG
CTCGAGCCCGACCCTGCTGCCGAGCCTCTGGTGCTGCCCCCCGTGGCT
GGTCGTTCTGTGGCTCTGTACGCTTCCGCCGACGAGGCTCGCAACGCT
CTGCCCCCCATCCCCCGTGTGATGTGGCCCCCTGGTTTCGGCGCTGCT
GAGACCGTCCTCGAGGGTTCCGACGGCACCCGTTTCGTGTTCGGTCAC
CACGGCGGTTCCGAGCGTCCCTCCGAGACCCAGGCTGGTCGCCAGCGC
CGTACCGCTGACGACCGTGAGCACGCTCTCGAGCTGGACGACTGGGAG
GTCGGCTGCGAGGACGCTTGGGACTCCGAAGAGGGTGGTGGCGACGAC
GGTGACGCTCCCGGCTCCTCCTTCGGTGTCTCCATCGTGTCCGTGGCT
CCCGGTGTCCTGCGTGACCGTCGTGTGGGTCTGCGTCCTGCTGTGAAG
GTGGAGCTGCTGTCCTCCTCTTCCTCTTCTGAGGATGAGGATGACGTG
TGGGGTGGTCGTGGTGGTCGCTCCCCCCCTCAGTCCCGTGGTTAA

About 800 ml of Sf9 cells, at about 2×10⁶ cells/ml in a 1 L shaker flask, were infected with recombinant baculovirus expressing IE62 at a multiplicity of infection (M01) of 1-3 infectious particles (pfu) per cell, incubated at 27 C with constant shaking, then harvested at about 64 hours post infection. The media was removed by low speed centrifugation and the cells lysed by 6000 rpm high shear homogenization (Silverson L4RT-A homogenizer) in 25 mM TrisCl pH 7.5, 250 mM NaCl. After centrifugation, the cell lysate was loaded to an anion exchange column (Fractogel TMAE, Merck KGaA, Germany) and eluted with 25 mM TrisCl pH 7.5, 500 mM NaCl. The elution was buffer exchanged into 25 mM NaPi pH 7.5, 375 mM NaCl with a Sephadex G25 (GE Healthcare) chromatography column. The flow through fraction from G25 column was load on a cation exchange column (Fractogel SO3−, Merck KGaA, Germany) and eluted with 25 mM NaPi pH 7.5, 625 mM NaCl. The elution from SO3− column became the final product of purified IE62 (FIG. 3 B; lane 8).

Results. Purified recombinant IE62 was >90% pure and contained both full length (150 KDa) and a small protein about 6 KDa (p6). IE62 and p6 were not separated by size exclusion chromatography and are present in approximately equal molar quantities. IE62 and p6 may be forming a heterodimer or other stable complex.

### Example 4

### Expression and Purification of gE/gI

Described is the expression and novel purification process for recombinant VZV gE/gI receptor heterodimer from Sf9 insect cells. gE and gI are surface glycoproteins and elicit neutralizing antibodies against VZV in man and immunized animals. A soluble form of the gE/gI heterodimer as produced in Sf9 insect cells and a purification process was developed to separate the secreted protein complex from host cell and baculovirus contaminants.

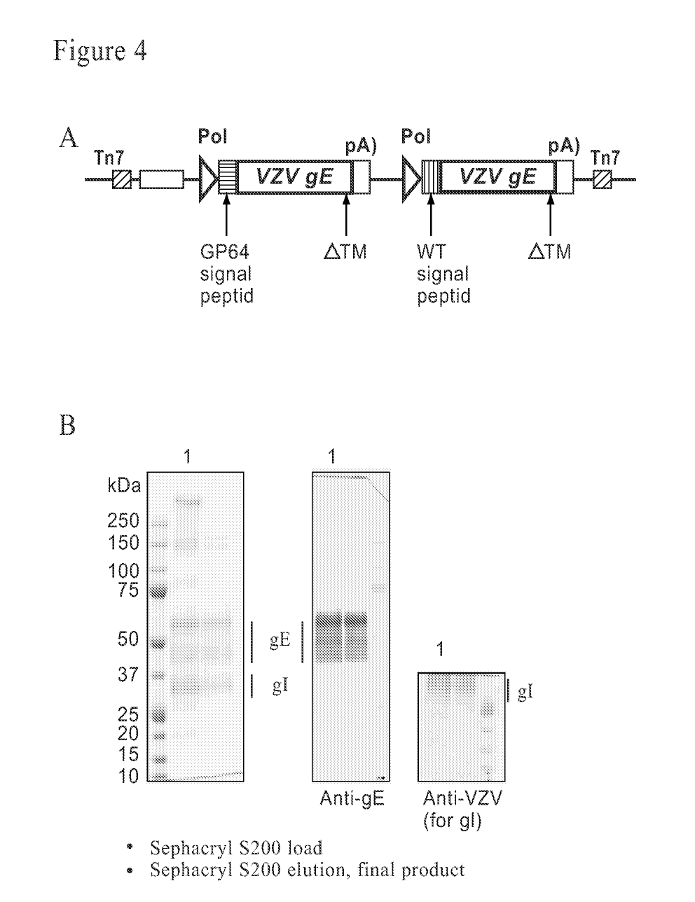

Methods. A baculovirus was engineered to express truncated, codon optimized genes of gE and gI from the Oka strain of VZV (see below). Both gE and gI have their transmembrane and carboxyl terminal domain removed. gI was made using the native gI signal peptide and the baculovirus GP64 signal peptide replaced the gE signal peptide sequence. The genes were synthesized (GeneArt, Germany) and cloned in tandem into a pFastBac1 vector with each gene under the control of a baculovirus polyhedrin promoter (FIG. 4A). The tandem gene cassette was transferred to an AcMNPV baculovirus Bacmid (Invitrogen), then Bacmid DNA was purified and used to transfect Sf9 insect cells. The resulting recombinant baculovirus was plaque-purified and virus stock prepared in Sf9 cells.

gE DeLtaTMCT
(SEQ ID NO: 17)
ATGGTGTCCGCTATCGTGCTGTACGTGCTGCTGGCTGCTGCTGCTCAC
TCCGCTTTCGCTCGTATCACCAACCCCGTGCGTGCTTCCGTGCTGCGT
TACGACGACTTCCACACCGACGAGGACAAGCTGGACACCAACTCCGTG
TACGAGCCCTACTACCACTCCGACCACGCTGAGTCCTCTTGGGTGAAC
CGTGGCGAGTCCTCCCGTAAGGCTTACGACCACAACTCCCCCTACATC
TGGCCCCGTAACGACTACGACGGTTTCCTCGAGAACGCTCACGAGCAC
CACGGTGTCTACAACCAGGGTCGTGGTATCGACTCCGGCGAGCGTCTG
ATGCAGCCCACCCAGATGTCCGCTCAGGAGGACCTGGGCGACGACACC
GGTATCCACGTGATCCCCACCCTGAACGGTGACGACCGTCACAAGATC
GTGAACGTGGACCAGCGCCAGTACGGTGACGTGTTCAAGGGTGACCTG
AACCCCAAGCCCCAGGGCCAGCGTCTGATCGAGGTGTCCGTGGAGGAG
AACCACCCCTTCACCCTGCGTGCTCCCATCCAGCGTATCTACGGTGTC
CGTTACACCGAGACCTGGTCCTTCCTCCCCTCCCTGACCTGCACCGGT
GACGCTGCTCCCGCTATCCAGCACATCTGCCTGAAGCACACCACCTGC
TTCCAGGACGTGGTGGTGGACGTGGACTGCGCTGAGAACACCAAGGAG
GACCAGCTGGCTGAGATCTCCTACAGGTTCCAGGGCAAGAAGGAGGCT
GACCAGCCCTGGATCGTGGTGAACACCTCCACCCTGTTCGACGAGCTG
GAGCTGGACCCCCCCGAGATCGAGCCCGGTGTCCTGAAGGTGCTGCGT
ACCGAGAAGCAGTACCTGGGCGTGTACATCTGGAACATGCGTGGTTCC
GACGGCACCTCCACCTACGCTACCTTCCTCGTGACCTGGAAGGGTGAC
GAAAAGACCCGTAACCCCACCCCCGCTGTGACCCCCCAGCCCCGTGGT
GCTGAATTCCATATGTGGAACTACCACTCTCACGTGTTCTCCGTGGGT
GACACCTTCTCCCTGGCTATGCACCTGCAGTACAAGATCCACGAGGCT
CCCTTCGACCTGCTGCTCGAGTGGCTGTACGTGCCCATCGACCCCACC
TGCCAGCCCATGCGCCTGTACTCCACCTGCCTGTACCACCCCAACGCT
CCCCAGTGCCTGTCCCACATGAACTCCGGTTGCACCTTCACCTCCCCC
CACCTGGCCCAGCGTGTGGCTTCCACCGTGTACCAGAACTGCGAGCAC
GCTGACAACTACACCGCTTACTGCCTGGGTATCAGCCACATGGAGCCT
TCCTTCGGTCTGATCCTGCACGACGGTGGCACCACCCTGAAGTTCGTG
GACACCCCCGAGTCCCTGTCCGGTCTGTACGTGTTCGTGGTGTACTTC
AACGGTCACGTGGAGGCTGTCGCTTACACCGTGGTGTCCACCGTGGAC
CACTTCGTGAACGCTATCGAGGAGCGTGGTTTCCCCCCCACCGCTGGC
CAGCCCCCTGCTACCACCAAGCCCAAGGAGATCACCCCCGTCAACCCC
GGCACCTCCCCTCTGCTGCGCTAA
gE DeltaTMCT
(SEQ ID NO: 18)
MVSAIVLYVL LAAAAHSAFA RITNPVRASV LRYDDFHTDE
DKLDTNSVYE PYYHSDHAES SWVNRGESSR KAYDHNSPYI
WPRNDYDGFL ENAHEHHGVY NQGRGIDSGE RLMQPTQMSA
QEDLGDDTGI HVIPTLNGDD RHKIVNVDQR QYGDVFKGDL
NPKPQGQRLI EVSVEENHPF TLRAPIQRIY GVRYTETWSF
LPSLTCTGDA APAIQHICLK HTTCFQDVVV DVDCAENTKE
DQLAEISYRF QGKKEADQPW IVVNTSTLFD ELELDPPEIE
PGVLKVLRTE KQYLGVYIWN MRGSDGTSTY ATFLVTWKGD
EKTRNPTPAV TPQPRGAEFH MWNYHSHVFS VGDTFSLAMH
LQYKIHEAPF DLLLEWLYVP IDPTCQPMRL YSTCLYHPNA
PQCLSHMNSG CTFTSPHLAQ RVASTVYQNC EHADNYTAYC
LGISHMEPSF GLILHDGGTT LKFVDTPESL SGLYVFVVYF
NGHVEAVAYT VVSTVDHFVN AIEERGFPPT AGQPPATTKP
KEITPVNPGT SPLLR
gI De1taTMCT
(SEQ ID NO: 19)
ATGTTCCTCATCCAGTGCCTGATCTCCGCTGTGATCTTCTACATCCAA
GTGACCAACGCTCTGATCTTCAAGGGTGACCACGTGTCCCTGCAGGTC
AACTCCTCCCTGACCTCCATCCTGATCCCCATGCAGAACGACAACTAC
ACCGAGATCAAGGGCCAGCTGGTGTTCATCGGCGAGCAGCTGCCCACC
GGCACCAACTACTCCGGCACCCTCGAGCTGCTGTACGCTGACACCGTC
GCTTTCTGCTTCCGTTCCGTGCAGGTGATCCGTTACGACGGTTGCCCC
CGTATCCGTACCTCCGCTTTCATCTCCTGCCGTTACAAGCACTCCTGG
CACTACGGTAACTCCACCGACCGTATCTCCACCGAGCCCGACGCTGGT
GTCATGCTGAAGATCACCAAGCCCGGTATCAACGACGCTGGCGTGTAC
GTGCTGCTGGTCCGTCTGGACCACTCCCGTTCCACCGACGGTTTCATC
CTGGGTGTCAACGTGTACACCGCTGGTTCCCACCACAACATCCACGGT
GTCATCTACACCTCCCCCTCCCTGCAGAACGGTTACTCCACCCGTGCT
CTGTTCCAGCAGGCTCGTCTGTGCGACCTGCCCGCTACCCCCAAGGGT
TCCGGCACCTCCCTCTTCCAGCACATGCTGGACCTGCGTGCTGGCAAG
TCCCTCGAGGACAACCCCTGGCTGCACGAGGACGTGGTGACCACCGAG
ACCAAGTCCGTGGTGAAGGAAGGTATCGAGAACCACGTGTACCCCACC
GACATGTCCACCCTGCCCGAGAAGTCCCTGAACGACCCCCCCGAGTAA
gI DeLtaTMCT
(SEQ ID NO: 20)
MFLIQCLISA VIFYIQVTNA LIFKGDHVSL QVNSSLTSIL
IPMQNDNYTE IKGQLVFIGE QLPTGTNYSG TLELLYADTV
AFCFRSVQVI RYDGCPRIRT SAFISCRYKH SWHYGNSTDR
ISTEPDAGVM LKITKPGIND AGVYVLLVRL DHSRSTDGFI
LGVNVYTAGS HHNIHGVIYT SPSLQNGYST RALFQQARLC
DLPATPKGSG TSLFQHMLDL RAGKSLEDNP WLHEDVVTTE
TKSVVKEGIE NHVYPTDMST LPEKSLNDPP E

About 800 ml of Sf9 cells, at about 2×10⁶ cells/ml in a 1 L shaker flask, were infected with recombinant baculovirus expressing C-terminal truncated gE and gI genes at a multiplicity of infection (MOI) of 1-3 infectious particles per ml (pfu), incubated at 27° C. with constant shaking, then harvested at 64 hours post infection. Cells were removed and the media collected by low speed centrifugation. gE/gI dimer in the medium was load to a Lentil Lectin affinity column and glycoproteins eluted with 500 mM Methyl-alpha-D-Mannopyranoside. The elution from lectin column was buffer exchanged into 25 mM TrisCl pH 8.0 50 mM NaCl with a Sephadex G25 column. The G25 chromatography protein peak was loaded on a Fractogel TMAE ion exchange column equilibrated with same buffer. gE/gI bound to the column and were eluted with a linear NaCl gradient. After concentration with an Amicon Ultra 10 kDa filter, the material was loaded on a Sephacryl S200 size exclusion column to remove high molecular weight contaminants. The final product was analyzed by SDS-PAGE and Western blot analysis (FIG. 4 B).

Results. The full length gE expressed in Sf9 insect cell is not glycosylated and does not bind to lentil lectin affinity resin, is insoluble, and remains cell-associated. Truncation of the C-terminal endodomain and transmembrane sequences results in secretion of a non-glycosylated and likely denatured form of the antigen (not shown). Co-expression of recombinant gE and gI in Sf9 cells produces gE/gI heterodimers that, due to the glycosylation of gI, binds to and can be purified by lectin affinity column chromatography. This soluble VZV glycoprotein complex could induce neutralizing and protective antibodies and be one component of a VZV vaccine. In addition, this heterdimers can be formulated with IE62 for a superior vaccine or antigenic formulation.

### Example 5

### Expression of gE in HEK293 Cells

Described is the expression of a secreted VZV gE glycoprotein in human HEK293 cells. VZV glycoproteins, for example gE, gI or gE/gI, gB can also be expressed in human or other mammalian cells or avian cell lines and are expected to be fully glycosylated. Purified glycoproteins could be used as one component of an VZV vaccine for example mixed with recombinant IE62 made in insect cells.

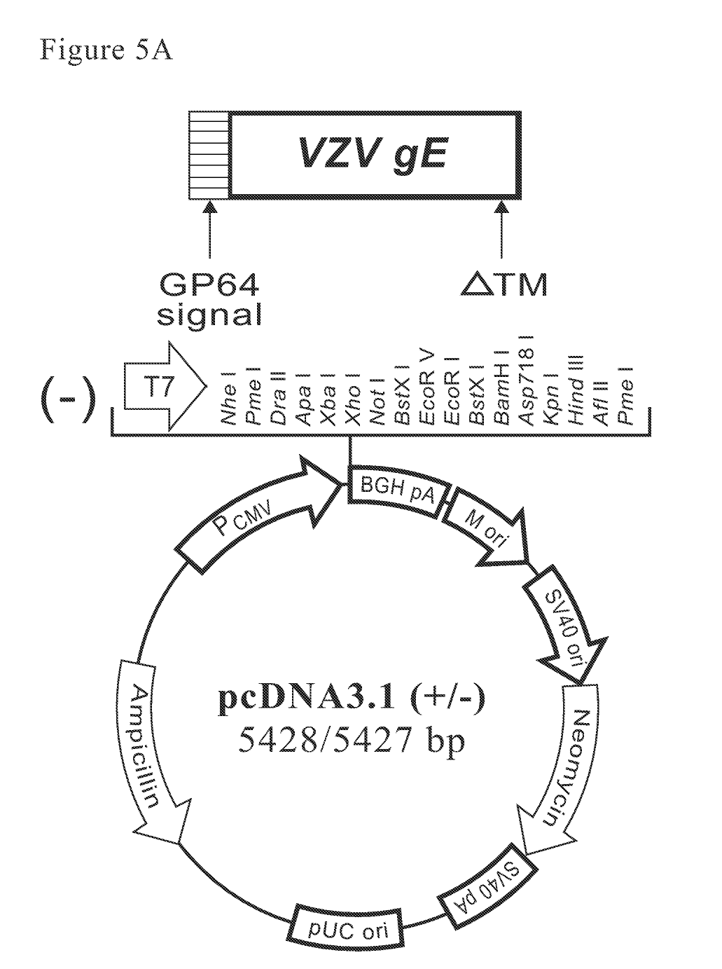

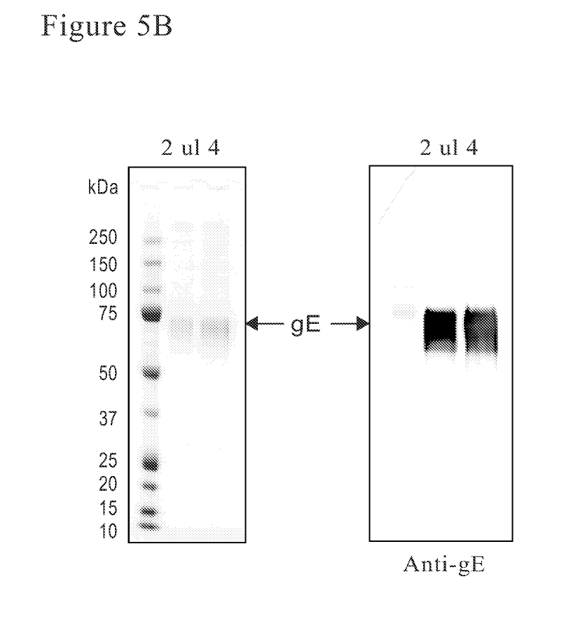

Methods. The coding sequence for VZV gE with GP64 signal peptide and removed transmembrane/carboxyl terminal domain was inserted into the pcDNA3.1 plasmid through Barn HI and Hind III sites (Invitrogen) (FIG. 5A). The final plasmid was used to transfect HEK293 freestyle cells (Invitrogen and as described). The HEK 293 freestyle cell culture medium was harvested 96 hours post transfection. The medium was load on a Lentil Lectin affinity column and eluted with 500 mM Methyl-alpha-D-mannopyranoside. The gE from a single lentil lectin column was >90% pure.

Results. gE protein was secreted into the culture medium since its transmembrane domain was removed (FIG. 5B). gE protein expressed in mammalian cell has authentic glycosylation pattern. The soluble gE expressed from HEK293 cells (˜70 kDa) was heavily glycosylated compared to non-glycosylated gE expressed from insect cells (˜60 kDa).

All patents, publications and patent applications herein are incorporated by reference to the same extent as if each individual patent, publication or cited patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1.-50. (canceled)

51. An antigenic formulation comprising a varicella zoster virus (VZV) gE/gI heterodimer and a VZV IE62 tegument protein.

52. The antigenic formulation of claim 51, wherein said gE/gI heterodimer is produced in Sf9 insect cells.

53. The antigenic formulation of claim 51, wherein the gene encoding said gE comprises SEQ ID NO: 17.

54. The antigenic formulation of claim 51, wherein said gE is comprised of SEQ ID NO: 18.

55. The antigenic formulation of claim 51, wherein said gE consists of SEQ ID NO: 18.

56. The antigenic formulation of claim 51, wherein said gE is expressed from a baculovirus vector.

57. The antigenic formulation of claim 51, wherein the gene encoding said gI comprises SEQ ID NO: 19.

58. The antigenic formulation of claim 51, wherein said gI is comprised of SEQ ID NO: 20.

59. The antigenic formulation of claim 51, wherein said gI consists of SEQ ID NO 20.

60. The antigenic formulation of claim 51, wherein said gI is expressed from a baculovirus vector.

61. The antigenic formulation of claim 51, wherein said IE62 tegument protein is produced in Sf9 insect cells.

62. The antigenic formulation of claim 51, wherein the gene encoding said IE62 tegument protein comprises SEQ ID NO: 16.

63. The antigenic formulation of claim 51, wherein said IE62 tegument protein is expressed from a baculovirus vector.

64. The antigenic formulation of claim 51, wherein said IE62 tegument protein is associated as a heterodimer with p6.

65. The antigenic formulation of claim 51, wherein said antigenic formulation further comprises an adjuvant or immune stimulator.

66. A method of using the antigenic formulation of claim 51 for eliciting protective immunity to VZV infection in a subject.

67. The method according to 66, wherein said subject is a human subject.

68. A purified VZV virus-like particle (VLP) comprising a VZV gE protein and at least one additional VZV protein.

69. The VLP of claim 63, wherein said additional VZV protein is gI.

70. The VLP of claim 64, further comprising a VZV tegument protein.
